(19) United States
(12) Patent Application Publication
(10) Pub. No.: US 2005/0239808 A1
(43) Pub. Date: Oct. 27, 2005

(54) Active substances for the treatment, diagnosis and prophylaxis of diseases in which abnormal protein structures occur

(76) Inventors: Thomas Schrader, Mettmann (DE); Detlev Riesner, Dusseldorf (DE); Petra Rzepecki, Trier (DE); Luitgard Nagel-Steger, Langenfeld (DE); Mark Wehner, Niedernhausen (DE); Christian Kirsten, Burscheid (DE); Oliver Molt, Hirschberg An Der Bergstrasse (DE); Reza Zadmard, Giessen (DE); Katja Aschermann, Wuppertal (DE)

Correspondence Address: CLARK & BRODY, 1090 VERMONT AVENUE, NW, SUITE 250, WASHINGTON, DC, 20005, US

(21) Appl. No.: 10/514,015

(22) PCT Filed: May 9, 2003
(86) PCT No.: PCT/DE03/01500

§ 371 Date: Jun. 24, 2005

(30) Foreign Application Priority Data
May 10, 2002 (DE) 102 21 052.7

(51) Int. Cl. A61K 31/522; A61K 31/4709; A61K 31/4196; A61K 31/416; C07D 473/02; C07D 43/02

(52) U.S. Cl. 514/263.21; 514/314; 514/383; 514/406; 544/276; 546/159; 548/262.8; 548/367.4

(57) ABSTRACT

The invention on hand refers to diseases associated with abnormal protein structures, including Alzheimer's disease, Creutzfeldt-Jakob's disease, BSE and other prion-associated diseases. It is the task of the invention on hand to provide new active agents preventing the formation of amyloid plaques and to dissolve existing plaques, such agents being convenient for therapy, diagnosis and prophylaxis of diseases that are associated with abnormal protein structures. This task is performed in terms of the current invention by heterocyclic or aromatic agents with a rigid structure and a donor-acceptor-donor pattern (DAD), the latter being formed by donors and acceptors of hydrogen bridge linkages which comply with the β-sheet structure of the peptide or protein and thus fit as binding partners. The heterocyclic or aromatic agents are available at least as dimers. They recognise peptides and proteins with a β-sheet structure, form stable complexes with them and inhibit their aggregation to β-amyloid plaques. Furthermore, the new active agents are able to dissolve β-amyloid plaques that already exist. Another task of the current invention is to provide methods for the synthesis of said heterocyclic or aromatic compounds.

In the past decades, the health situation in developed countries has considerably improved, resulting in an increased life expectancy. Due to the increased ratio of elderly people in the total population, diseases that are conditional to age are more widespread today, than several years ago.

Dementia belongs to a group of diseases, which are more frequent amongst the elderly. One such dementia, Alzheimer's disease, has particular impact. A disposition associated with a profound impairment of cognitive capacities, Alzheimer's disease affects from 830,000 to 1.1 million people in Germany over the age of 65. Approximately 1.2% of all 65 year olds and 34.6% of all people over 90 suffer from Alzheimer's. From the age of 65, the quantity of affected individuals within an age cohort doubles nearly every five years.

The neurobiological characteristics of Alzheimer's disease involve the conversion of normal α-helical protein structures into abnormal β-sheet structures (β-amyloid plaques, β-AP) the excessive loading of τ-proteins of the cytoskeleton with phosphate groups and the degeneration of muscarinic cholinergic neurones. β-AP deposit between neurones causing profound impairment to the oxygen and energy supply of the brain, finally resulting in neurone death. τ-proteins are standard parts of the cytoskeleton. An excessive loading of these proteins with phosphate groups causes dysfunction in stabilisation and transport processes, which leads to cell death. Degeneration of the muscarinic cholinergic neurones is accomplished by a chronic lack of the neurotransmitter acetylcholine, resulting in a radically reduced signal transmission between neurones.

These pathophysiological processes are implicated in decreasing mental capacities and increasing problems in coping with everyday life. During the final stages of the disease the patients are entirely dependent on carers. The most important factor in developing Alzheimer's disease is old age; a genetic predisposition may add a further risk. Early diagnosis is presently difficult; the detection of typical β-amyloid plaques is not possible until death. Reliable laboratory tests for diagnosis do not yet exist. Graphical representations of the regional blood circulation and metabolism in the brain, and examinations of the behavioural symptoms and cognitive capacities are useful indicators, but reliable diagnosis is still not possible in the early stages of the disease.

Medical therapy currently focuses primarily on acetylcholine esterase inhibitors that decelerate or prevent the degradation of acetylcholine. Alternatively or complementary, phytotherapeutics (St. John's Wort, Cat's claw and others) as well as several non-steroidogenic antiphlogistics (e.g. Ibuprofen) are applied, however the mode of action of these drugs is unknown.

### DESCRIPTION AND TECHNICAL STATE OF THE ART

Characteristic for Alzheimer's disease and other forms of dementia are the formation of abnormal β-sheet structures of peptides and proteins (β-amyloidogenic peptides or plaques, βAP) as well as the degeneration of muscarinic cholinergic neurones. At the beginning of the disease, so-called amyloid precursor proteins (APP) are formed, which are later cleaved by secretase. The cleavage products interfold themselves finally into the characteristic β-amyloid plaques. Specific mutations of the apolipoprotein E (ApoE) appear to either facilitate or impede the onset of Alzheimer's.

The current focus of medical treatment is on antidementia pharmaceuticals. Centrally functioning medications are designated as antidementia pharmaceuticals, which can improve highly integrative functions such as memory, concentration, learning aptitude, judgement capacity, as well as orientation and vigilance. A standardised mechanism does not exist and only a few of the pharmaceuticals can fulfil the standard guidelines of efficacy.

Cognitive functions of people suffering from Alzheimer's are said to be improved through the use of R-Ibuprofen, Melatonin, as well as supplements of St. John's Wort (Hyperforicum perforatum L) and Cat's claw (Uncaria tomentosa). The mode of action and the success of these supplements in treating Alzheimer's are unclear. With the acetylcholine esterase inhibitors, a new substance group has been introduced in the last few years, which have a clearly defined mode of action and can delay the progression of cognitive deficits for up to 12 months (T. Kratzsch (2002), Neurotransmitter 3: 52-55). Included in these are Cognex®, Aricept® und Exelon®. The application of pharmaceuticals from this substance group is associated with severe side effects however, such as nausea, gastrointestinal problems, vertigo, sleep disorders and a slowed reaction capacity.

U.S. Pat. No. 6,313,268B1 describes β-secretase, which is suitable for cleaving APP at a specific point. This approach is probably therapeutically inadequate, since the cleavage of APP by secretase also occurs during the disease and does not prevent the aggregation of the fragments to β-amyloid plaques. U.S. Pat. No. 5,948,763A, U.S. Pat. No. 6,277,826B1, U.S. Pat. No. 6,303,567B1, U.S. Pat. No. 6,319,498B1 and U.S. Pat. No. 6,277,826B1 describe peptides which bind to βAP and not only inhibit the formation of abnormal β-sheet structures but also the regeneration of βAP. With these substances, as with all peptides, an allergy risk can not be ruled out. U.S. Pat. No. 6,277,874 describes dyes, organic salts and dextrane, which specifically inhibit the toxic effect of ApoE. U.S. Pat. No. 6,323,218B1 describes the application of chelating agents and clioquinol, with whose help the Aβ-based production of radical oxygen species shall be prevented or inhibited. The formation of β-sheet structures is however not prevented in this way. In WO 00/76988A1, WO 00/76969A1 and WO 01/83425A1 substituted rhodanine, isoindoline and aminoindane are described, which can be used in the inhibition of plaque formation and therapy of Alzheimer's disease. U.S. Pat. No. 6,329,356B1 describes polysubstituted ω-phosphonate alkylcarbylic acid, which modulates the interaction between βAP and membrane components. With this therapy however, potential allergenic effects of the alkylcarbylic acid have to be taken into consideration. EP 05447799 describes substituted oxadiazole and thiadiazole compounds which function as muscarinic cholinergic agonists. Numerous pathological processes are closely related to the formation of β-sheet structures and the following protein aggregation. Apart from Alzheimer's disease, Creutzfeldt-Jakob disease, BSE and other prion diseases should be mentioned in this context.

The task of the invention on hand is to make new active agents available which prevent the formation of amyloid plaques and dissolve existing plaques, said active agents being applicable for the treatment, diagnosis and prophylaxis of diseases related to abnormal protein structures.

This task is performed in accordance with the terms of the current invention; by heterocyclic or aromatic agents with a rigid structure and a donor-acceptor-donor pattern (DAD) The latter was formed by donors and acceptors of hydrogen bridge linkages, which comply with the β-sheet structure of the peptide or protein and thus fit as binding partners. The heterocyclic or aromatic agents are available at least in the form of dimers. They recognise peptides and proteins with a β-sheet structure, form stable complexes with them, and avoid their aggregation to β-amyloid plaques.

Furthermore, the new active agents are able to dissolve β-amyloid plaques that already exist. Therapy, diagnostics and prophylaxis of diseases that are related to abnormal protein structures represent a convenient commercial application of said heterocyclic and aromatic agents.

Another task of the invention is to provide synthesis methods for the production of said heterocyclic or aromatic agents. This task is performed in terms of the invention by synthesis instructions 1 and 2 (see below).

Synthesis instruction 1 is a process represented by a) the conversion of a ring N-1-protected heterocyclic amine (protecting group: acetal, acyl, silyl, benzyl; in case of an aminopyrazole preferably Boc) with a carboxylic acid dihalogenide yielding the corresponding diacyl-bridged compound and b) subsequent cleavage of the N-1 protecting group. Asymmetrical diacyl-bridged compounds of said type can be obtained by sequential conversion of two different heterocyclic amines with asymmetrical linkers, i.e. a carboxylic acid halogenide at one end and an ester group at the other end. If the heterocycle represents a diamine, under a) (above) the amine which is both protected in the ring N-1 and monoprotected regarding the amino substituents is converted. Subsequently, the amino protecting groups are cleaved off under mild conditions according to b)(above), concomitantly maintaining the ring N-1 protecting group (e.g. cleavage of Fmoc while maintaining Boc). Then, under c) a new diacyl bridge is introduced under the same conditions as described under a) and under d) the ring N-1 protective group is finally cleaved off (e.g. Boc with TFA). Synthesis instruction 2 is a process represented by six different modules, which can be coupled to each other in any possible order: Two of the modules consist of a ring N-1 protected heterocyclic amino carboxylic acid ester and a ring N-1 as well as amino protected heterocyclic amino carboxylic acid. Two other modules consist of N- or C-protected α- or β-amino acids. The last two modules form the terminal groups and consist of any amine or any carboxylic acid. Linking of the modules may be performed blockwise or sequentially, namely from the N- to the C-terminus or inversely. In order to avoid racemisation, build up of the peptide-like oligomer in the direction of C-terminus to N-terminus is recommended, if amino acids are present. In this case, the first module already carries a terminal group; if amino acids are present, this terminal group may be any amine. Finally, the other terminal group is linked (in the presence of amino acids, this terminal group may be any carboxylic acid), and lastly all protecting groups are cleaved off again. Additionally, the presence of amino acids requires reaction conditions that do not allow racemisation. Therefore, protecting groups derived from peptide synthesis are preferably applied. Ring N-1 of aminopyrazole carboxylic acids are best provided with a p-methoxybenzyl group (PMB) protection group, which should be cleaved off in the last step with warm trifluoroacetic acid (TFA).

Today, state of the art techniques include knowledge of the active agents involved in diagnosis, prophylaxis and therapy of diseases, which are associated with the formation of abnormal protein structures. However these applications do not admittedly always provide satisfying results. For example, supplements with St. John's Wort or Cat's claw should improve cognitive functions, but their mode of action and therapeutic success are unknown. R-Ibuprofen use is associated with the same problems. In addition, this drug has to be applied in comparatively high doses and toxic side effects cannot be excluded. Contrarily, the metabolism of the acetylcholine esterase inhibitors used for Alzheimer's therapy is clearly defined; however taking these supplements causes severe side effects such as nausea, gastrointestinal disorders, vertigo, sleeping disorders and a slowed reaction capacity. The application of peptides that clearly bind to distinct areas of the βAP, implies a high risk of causing allergies. Several dyes should specifically inhibit the toxic effects of apolipoprotein E (ApoE), and clioquinol should inhibit the βAP-induced formation of radical oxygen species. However, the formation of toxic products of ApoE and/or radical oxygen species merely represent side effects of the Alzheimer's disease. Thus, the application of the aforementioned substances barely influences the formation of the characteristic and pathological amyloid plaques. According to WO 00/76988A1, WO 00/76969A1 and WO 01/83425A1, several heterocyclic or aromatic substances inhibit the formation of, and dissolve pre-existing βAP. The nature of the interaction between the heterocyclic or aromatic compounds and the βAP structures remains unknown. As these compounds do not feature a DAD pattern, a specific DAD interaction with the pathogenic peptides is excluded. Substituted oxadiazole and thiadiazole compounds, as disclosed in EP 0544779, act as muscarinic cholinergic agonists. This means the drugs do not affect pathogenic βAP structures but the degenerated neurones.

None of the known compounds feature an elucidated form of interaction with the pathogenic processes related to Alzheimer's disease. Nor are they specifically directed against pathogenic βAP structures.

The active agents based on the current invention are characterised by the fact that they recognise and bind to β-sheet structures. Their particular DAD structure precisely fits the β-sheet structure of peptides and proteins and fulfils the lock-and-key principle of molecular recognition, whereby DAD stands for hydrogen bridge linkages donor-acceptor-donor. The term donor-acceptor-donor was introduced in research carried out by Jorgensen who systematically examined the order of donors and acceptors in host-guest systems and thereupon created the concept of secondary interaction (W. L. Jorgensen and J. Pranata, J. Am. Chem. Soc. 1990, 112, 2008). The β-sheet conformation of naturally occurring peptides shows a strongly alternating order of acceptor (C═O group), donor (NH group) acceptor (next C═O group), continuing through the whole peptide. Therefore, in an ideal case, β-sheet ligands need a complementary sequence of donor-acceptor-donor in order to bind all haptic groups of the peptide via hydrogen bridge linkages. The terminal atoms that bind in the peptide (O1, H, O2) are approximately in line with a O1-H distance of about 3.5-4.0 Å and H-O2 distance of about 2.6-2.9 Å. Thus, terminal atoms in the receptor must be approximately in line with a mutual distance similar to that in the peptide as well. This condition is fulfilled by the active agents according to the present invention: 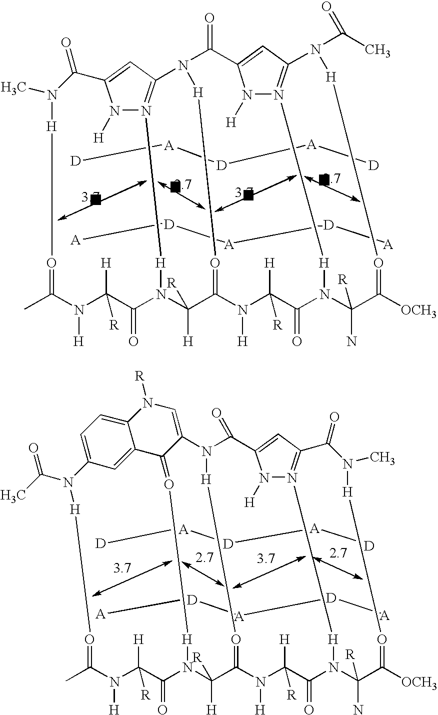
Complementary ligand-peptide structures (DAD-ADA). Distances are given in Å.

Hence, the active agents based on the current invention may be applied to treatment, diagnosis and prophylaxis of diseases related to β-sheet formation followed by abnormal protein aggregation. Amongst these are prion diseases such as transmissible spongiform encephalopathies, Alzheimer's disease, Creutzfeldt-Jakob's disease (CJD), the new variant of Creutzfeldt-Jakob's disease (nvCJD), Kuru disease, Gerstmann-Sträusler-Scheinker's syndrome, Fatal Familial Insomnia (FFI) and Scrapies.

Active agents based on the current invention are any such heterocyclic or aromatic agents and their oligomers which bind to proteins and peptides with a β-sheet structure following the donor-acceptor-donor principle and which are available at least in the form of dimers. The individual heterocyclic or aromatic units within the active agents based on the current invention are coupled covalently to each other via aliphatic, aromatic, heteroaromatic, amino acid or short-chain peptide groups. These functional groups connecting the heteroaromatic and aromatic units are named “spacers”. The active agents based on the current invention may carry hydrophobic substituents in order to enhance [symbol] n agent's affinity for the protein and/or charged or polar groups on one or both ends to enhance the affinity for the C- or N-terminus of the protein or peptide to be bound. The present invention includes all such enantiomers, diastereomers and racemates of the above-mentioned active agents that are available by combination of the aforementioned heterocyclic compounds, spacers and substituents.

The following heterocyclic and aromatic basic elements fulfil the task to satisfy the donor-acceptor-donor principle, e.g.:

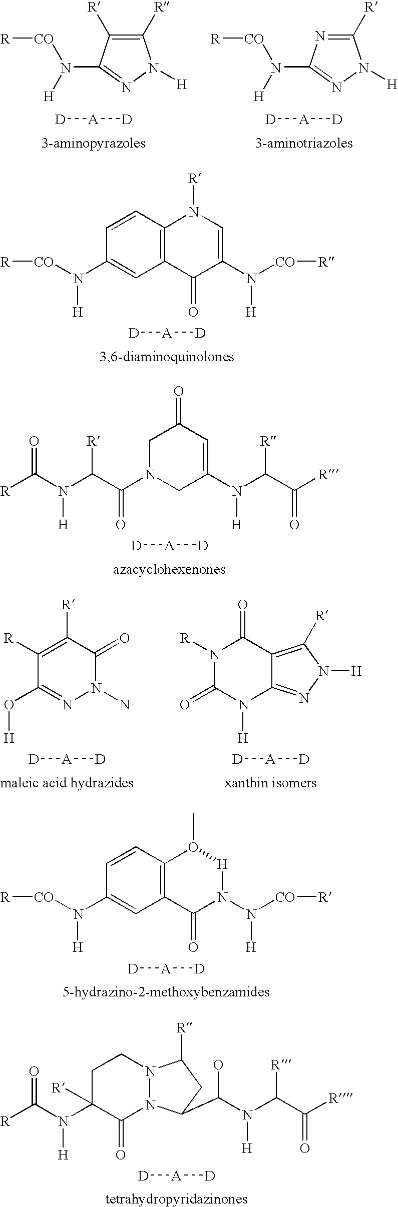

Coupling to oligomers is thereby established via rests R-groups up to R—R″′′. All R-groups except one per module concomitantly may carry solubility enhancing groups. The heterocyclic and aromatic active agents are produced via two synthesis methods, both based on the current invention. The first synthesis method provides diacyl-bridged heterocyclic or aromatic oligomers of type I:

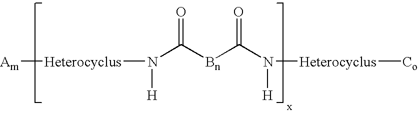

Where A, C=Rest: linear, branched or cyclic alkyl, alkenyl or alkynyl with or without OH substituents or halides, phenyl, phenylalkyl, phenylalkenyl, phenylalkynyl, phenylcycloalkyl, phenylcycloalkenyl, phenylcycloalkynyl, cycloalkyl-alkyl, cycloalkyl-alkenyl, cycloalkyl-alkynyl, heterocyclic alkyl, heterocyclic alkenyl, heterocyclic alkynyl, acyl, aryl, aryloxy, heteroaryl, heteroaryloxy, aroyl, benzyl, (aryl) alkyloxycarbonyl, linear or branched alkoxyalkyl with or without OH substituents, polyethoxy-alkyl, polyethoxy-alkenyl, polyethoxy-alkynyl, polyethoxy-cycloalkyl, polyethoxy-cycloalkenyl, polyethoxy-cycloalkynyl, polyethoxy-aryl, polyethoxy-alkyl-aryl, polyethoxy-heterocycloalkyl, polyethoxy-heterocycloaryl, primary, secondary, tertiary or quaternary ammonium, amino-alkyl, amino-alkenyl, amino-alkynyl, amino-cycloalkyl, amino-alkyl-cycloalkyl, amino-cycloalkyl-alkyl, amino-phenyl, amino-alkyl-phenyl, amino-phenyl-alkyl, all hydroxylamines, all cyano compounds, all nitriles and isonitriles, all halides, formyl, alkanal, alkenal, alkynal, cycloalkenal, benzyl carbaldehyde, heteroaryl-carbaldehyde, benzyl-alkyl-carbaldehyde, heteroaryl-carbaldehyde, aliphatic heteroalkyl-alkenal (-alkenyl-alkenal, alkynyl-alkenal), alkanone, alkenone, alkynone, cycloalkyl-alkanone, dicycloalkanone, arylalkanone, heteroaryl-alkanone, nitro, alkylsulfoxy, alkylsulfonyl, CONH₂, CONHR, CONR₂, all imines, all oximes, all hydrazones, CH═NOR, thio, thio-alkyl, thio-alkenyl, thio-alkynyl, thio-cycloalkyl, thio-alkyl-cycloalkyl, thio-cycloalkyl-alkyl, thio-phenyl, thio-alkyl-phenyl, thio-phenyl-alkyl, alkylthio, halide, hydroxy, hydroxy-alkyl, hydroxy-alkenyl, hydroxy-alkynyl, hydroxy-cycloalkyl, hydroxy-alkyl-cycloalkyl, hydroxy-cycloalkyl-alkyl, hydroxy-phenyl, hydroxy-alkyl-phenyl, hydroxy-phenyl-alkyl, alkoxy, alkoxycarbonyl, alkyl-, alkenyl, alkynyl-, aryl-, heteroalkyl-, heteroaryl-carboxylic acids, all esters and carboxylic amides thereof, linear or branched alkylsulphonate, alkenylsulphonate, alkynylsulphonate, linear or branched alkylbenzenesulphonate, alkenylbenzenesulphonate, alkynylbenzenesulphonate, aminosulphonyl-alkyl, aminosulphonyl-alkenyl, aminosulphonyl-alkynyl, aminosulphonyl-cycloalkyl, aminosulphonyl-cycloalkenyl, aminosulphonyl-cycloalkynyl, linear or branched alkyl-sulphonamide, alkenyl-sulphonamide, alkynyl-sulphonamide, cycloalkyl-sulphonamide, cycloalkenyl-sulphonamide, cycloalkynyl-sulphonamide, phenyl-sulphonamide, heterocyclo-sulphonic acid, heterocyclo-sulphonamide, heterocyclo-alkyl-sulphonic acid, heterocyclo-alkyl-sulphonic acid, heterocyclo-alkenyl-sulphonic acid, heterocyclo-alkenyl-sulphonamide, heterocyclo-alkynyl-sulphonic acid, heterocyclo-alkynyl-sulphonic acid, aryl-sulphonic acid, aryl-sulphonamide, aryl-alkyl-sulphonamide, aryl-alkyl-sulphonamide, aryl-alkenyl-sulphonic acid, aryl-alkenyl-sulphonamide, aryl-alkynyl-sulphonic acid, aryl-alkynyl-sulphonamide, alkyl-, alkenyl, alkynyl-, aryl-, heteroalkyl-, heteroaryl-phosphonic acids as well as all esters and amides thereof, alkyl-, alkenyl, alkynyl-, aryl-, heteroalkyl-, heteroaryl-phosphinic acids as well as all esters and amides thereof.

B=spacer (represents no atom, linear or branched alkyl, alkenyl, alkynyl with or without OH substituents or halides, aryl, aryl(di)oxy, heteroaryl, heteroaryl(di)oxy, benzyl, xylyl, linear or branched alkoxyalkyl with or without OH substituents, oligo(ethyleneglycol), amino, alkylamino, CH═NR, CH═NOR, alkoxy or hydrazino group, wherein B and n may be different in any unit x.
m, o=1-3
n=0-3
X=1-5

### EXAMPLES

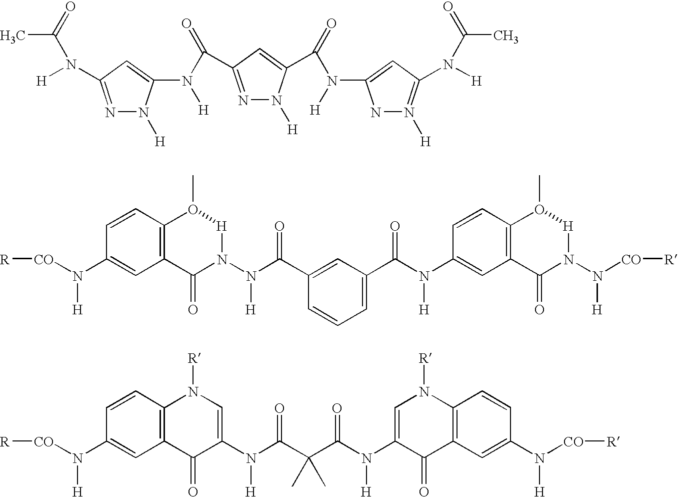

The production of the active agents of type I is carried out according to synthesis instruction 1, based on the current invention:

In order to produce symmetrical diacyl-bridged heterocyclic dimers ring N-1 protected heterocyclic amines and to produce the homologous tri- up to oligomers heterocyclic diamines are utilised. Heterocyclic diamines need to be both protected at ring N-1 and monoprotected regarding the amino substituents via an orthogonal protection group. Appropriate protection groups for the heterocycle (amine or diamine) are most notably Boc, Z, Fmoc or acetals, whereby the protection group is to stand in an orthogonal position if it protects the amino substituent of the diamine. In the case of an aminopyrazole, Boc is preferentially used.

In order to produce a symmetrical product, 2 eq of the ring N protected amine or the mono-protected diamine and 3 eq of a sterically demanding base (e.g. triethylamine, Hünig's base, N-methyl morpholine) are dissolved in an organic solvent, converted with 1 eq of a diacid chloride and stirred overnight at room temperature. After having carried out the extraction according to methods known to the expert and, if necessary, purification by means of column chromatography, yields the ring N protected product. In order to obtain diacyl-bridged heterocyclic dimers, the ring N-1 atom is deprotected according to methods known from literature.

In order to obtain the homologous tri- up to oligomers, the amino-protecting group of the mono-protected diamine is deprotected according to methods known from literature. Subsequently, the deprotected dimer obtained in this way is converted with a diacyl compound again. This reaction order is continued and yields the respective trimers up to oligomers after deprotection of the ring.

In addition to the symmetrical product, the production of asymmetrical dimers up to oligomers using different heterocyclic compounds is also possible. To proceed, 1 eq of the ring N protected amine or the mono-protected diamine and 1.5 eq of a sterically demanding base (e.g. triethylamine, Hünig's base, N-methyl morpholine) are dissolved in an organic solvent and converted overnight with 1 eq of a diacyl compound under stirring and ice cooling conditions. The diacyl compound carries a acid chloride on one end and a carboxylic acid ester on the other one. Subsequently, an excess of another ring N protected heterocyclic amine and 1.5 eq of a sterically demanding base (e.g. triethylamine, Hünig's base, N-methyl morpholine) are added and stirred overnight under heating to cause reflux. Subsequent deprotection of the ring, according to methods known to the expert, and chromatographic purification, yield the respective product. When dealing with an amine that is ring N protected and monoprotected by an orthogonal heterocyclic diamine protection group, the reaction sequence may be repeated as many times as desired, yielding asymmetrical oligomers.

The second synthesis instruction based on the current invention yields peptide-like bridged heterocyclic oligomers of type II: 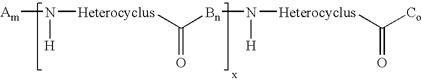
Wherein
A, C=rest (linear, branched or cyclic alkyl, alkenyl or alkynyl with or without OH substituents or halides, phenyl, phenylalkyl, phenylalkenyl, phenylalkynyl, phenylcycloalkyl, phenylcycloalkenyl, phenylcycloalkynyl, cycloalkyl-alkyl, cycloalkyl-alkenyl, cycloalkyl-alkynyl, heterocyclic alkyl, heterocyclic alkenyl, heterocyclic alkynyl, acyl, aryl, aryloxy, heteroaryl, heteroaryloxy, aroyl, benzyl, (aryl)alkyloxycarbonyl, linear or branched alkoxyalkyl with or without OH substituents, polyethoxy-alkyl, polyethoxy-alkenyl, polyethoxy-alkynyl, polyethoxy-cycloalkyl, polyethoxy-cycloalkenyl, polyethoxy-cycloalkynyl, polyethoxy-aryl, polyethoxy-alkyl-aryl, polyethoxy-heterocycloalkyl, polyethoxy-heterocycloaryl, primary, secondary, tertiary or quaternary ammonium, amino-alkyl, amino-alkenyl, amino-alkynyl, amino-cycloalkyl, amino-alkyl-cycloalkyl, amino-cycloalkyl-alkyl, amino-phenyl, amino-alkyl-phenyl, amino-phenyl-alkyl, all hydroxylamines, all cyano compounds, all nitriles and isonitriles, all halides, formyl, alkanal, alkenal, alkynal, cycloalkenal, benzyl carbaldehyde, heteroaryl-carbaldehyde, benzyl-alkyl-carbaldehyde, heteroaryl-carbaldehyde, aliphatic heteroalkyl-alkenal (-alkenyl-alkenal, alkynyl-alkenal), alkanone, alkenone, alkynone, cycloalkyl-alkanone, dicycloalkanone, arylalkanone, heteroaryl-alkanone, nitro, alkylsulfoxy, alkylsulfonyl, CONH₂, CONHR, CONR₂, all imines, all oximes, all hydrazones, CH═NOR, thio, thio-alkyl, thio-alkenyl, thio-alkynyl, thio-cycloalkyl, thio-alkyl-cycloalkyl, thio-cycloalkyl-alkyl, thio-phenyl, thio-alkyl-phenyl, thio-phenyl-alkyl, alkylthio, halide, hydroxy, hydroxy-alkyl, hydroxy-alkenyl, hydroxy-alkynyl, hydroxy-cycloalkyl, hydroxy-alkyl-cycloalkyl, hydroxy-cycloalkyl-alkyl, hydroxy-phenyl, hydroxy-alkyl-phenyl, hydroxy-phenyl-alkyl, alkoxy, alkoxycarbonyl, alkyl-, alkenyl, alkynyl-, aryl-, heteroalkyl-, heteroaryl-carboxylic acids, and all esters and carboxylic acid amides thereof, linear or branched alkylsulphonate, alkenylsulphonate, alkynylsulphonate, linear or branched alkylbenzenesulphonate, alkenylbenzenesulphonate, alkynylbenzenesulphonate, aminosulphonyl-alkyl, aminosulphonyl-alkenyl, aminosulphonyl-alkynyl, aminosulphonyl-cycloalkyl, aminosulphonyl-cycloalkenyl, aminosulphonyl-cycloalkynyl, linear or branched alkyl-sulphonamide, alkenyl-sulphonamide, alkynyl-sulphonamide, cycloalkyl-sulphonamide, cycloalkenyl-sulphonamide, cycloalkynyl-sulphonamide, phenyl-sulphonamide, heterocyclo-sulphonic acid, heterocyclo-sulphonamide, heterocyclo-alkyl-sulphonic acid, heterocyclo-alkyl-sulphonamide, heterocyclo-alkenyl-sulphonic acid, heterocyclo-alkenyl-sulphonamide, heterocyclo-alkynyl-sulphonic acid, heterocyclo-alkynyl-sulphonamide, aryl-sulphonic acid, aryl-sulphonamide, aryl-alkyl-sulphonic acid, aryl-alkyl-sulphonamide, aryl-alkenyl-sulphonic acid, aryl-alkenyl-sulphonamide, aryl-alkynyl-sulphonic acid, aryl-alkynyl-sulphonamide, alkyl-, alkenyl, alkynyl-, aryl-, heteroalkyl-, heteroaryl-phosphonic acids and all esters and amides thereof, alkyl-, alkenyl, alkynyl-aryl-, heteroalkyl-, heteroaryl-phosphinic acids and all esters and amides thereof)
B represents an amino group (NH) and/or any natural or unnatural α- or β-amino acid in their (D)- or (L)-configuration, wherein the said amino acid may vary in any unit x.
m, o=1-5
n=0-5
x=1-5

### EXAMPLES

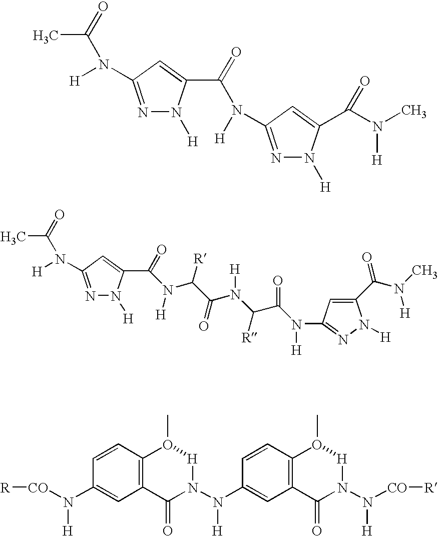

The production of the active agents of type II, based on the current invention, is to be prepared according to synthesis instruction 2, based on the current invention.

For representation the following modules are produced which are subjected to a sequential peptide synthesis into a solution or a solid phase reaction:
1. Heterocyclic amino acids carrying a ring N protective group as well as an N-terminal orthogonal protective group. In the case of 3-aminopyrazole-5-carboxylic acid, ring N is preferably protected by PMB, according to methods known to the expert, and the N terminus is masked as a nitro group or with an Fmoc group if it deals with a solid phase reaction.
2. Ring N as well as at C-terminus protected heterocyclic amino acids. In case of 3-aminopyrazole-5-carboxylic acid, the ring N is preferably protected by PMB according to known methods, and the C terminus is made functional as a methyl ester via acid-catalysed esterification.
3. Natural or unnatural amino acids which are protected at the N-terminus and, if necessary, also at the side chain. The N-terminus is protected by protecting groups known from the peptide chemistry, such as Boc, Fmoc and Z.
4. When dealing with the last link of a synthesis in the direction from the C-terminus to the N-terminus, an acyl protective group is preferred (rest A).
5. Natural or unnatural amino acids which are protected at the C-terminus and, if necessary, also at the side chain. The C-terminus is protected by protecting groups known from the peptide chemistry, such as tBu.
6. When dealing with the last link of a synthesis in the direction from the N-terminus to the C-terminus, an ester or amide protective group is preferred (rest C).

These modules may be combined in any desired manner. For this purpose, 1 eq of the acid (Either a heterocyclic amino acid carrying a ring N-1 protective group and an orthogonal protective group at the N-terminus or a natural or unnatural amino acid carrying a protective group at the N-terminus and, if necessary, a protective group a the side chain) and 1 eq of the amine (Either a heterocyclic amino acid carrying protective groups at ring N and at C-terminus or a natural or unnatural amino acid carrying a protective group at the C-terminus and, if necessary, a protective group at the side chain) are dissolved in an organic solvent and coupled under peptide coupling conditions as described in technical literature.

Most preferably, the following peptide coupling reagents are applied: DCI, PyClop, HBTU, TBTU, HATU, T3P, PyBop, BopCl or 3-chloro-1-methylpyridiniumiodide, all of which are added in quantities of 1.3-1.5 eq and in some cases, together with 2.5 eq HOBt or HOAt. For example, triethylamine, Hünig's base or N-methyl morpholine may act as useful bases. The choice of the coupling reagent depends on the particular modules used. For example, the coupling of two suitably protected 3-aminopyrazole-5-carboxylic acids best succeeds when the mixture is refluxed and PyClop and Hünig's base in dichloromethane are used as coupling reagents (see practical embodiment 2 below, example 5). Amino acids at risk of racemisation are converted according to standard peptide coupling instructions. Either the C-terminal or the N-terminal protective group can then be cleaved off and the compound formed in this manner is converted with another module according to the method described above, following either a linear or a convergent synthesis strategy. N-terminal end groups may be protected either with a peptide coupling reagent (when dealing with an acyl-protected amino acid), or they may be induced via the anhydride or acid chloride of the respective carboxylic acid.

C-terminal end groups may be protected with a peptide-coupling reagent (when dealing with an ester-protected amino acid) or via reamidation via reaction of a C-terminal ester with an excess of amine under reflux conditions.

In the case of a solvent synthesis, after each coupling step a purification of the intermediate by means of column chromatography is carried out.

When all the coupling steps are completed, the groups protecting the heterocycles are cleaved off in one step. For example, the PMB protective group is cleaved off by refluxing the compound in absolute TFA under argon atmosphere (see practical embodiment 3, example 10). Thereafter, the product is obtained via purification by means of column chromatography.

### Practical Embodiment 1: Production of Diacyl-Bridged Heterocyclic Dimers Type I

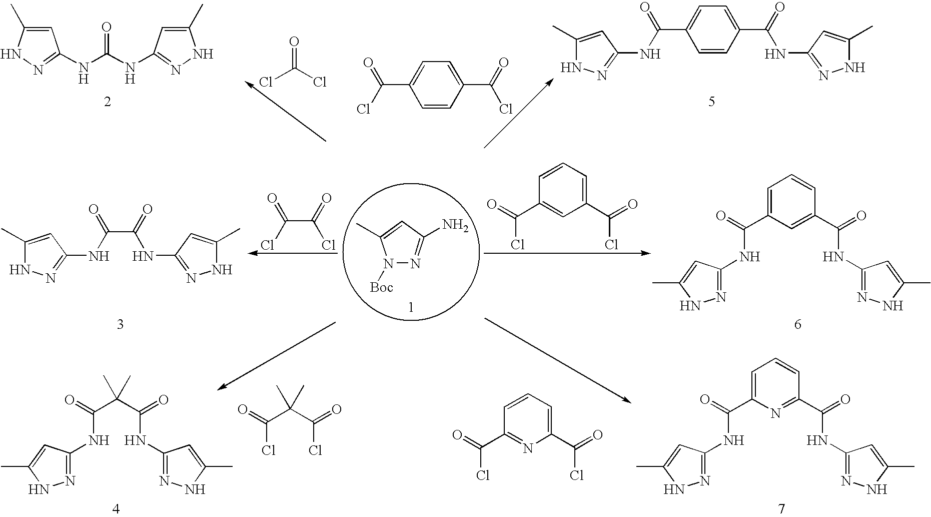 General Instruction A Pertaining to Practical Embodiment 1: Representation of Boc-Protected Dimers

2 equivalents of N-1-Boc-3-aminopyrazole and 3 equivalents of NEt₃ were dissolved in CH₂Cl₂ ((abs.)). 1 equivalent of the diacid chloride was added at 0° C. and the mixture was stirred overnight at room temperature. The solvent was evaporated and the remaining residue shaken out with acetate ester and 1N HCl solution, saturated NaHCO₃, saturated NaCl and distilled water. The organic phase was dried over magnesium sulphate, filtered and evaporated. The further purification will be described together with the respective substances.

### General Instruction B Pertaining to Practical Embodiment 1: Splitting Off the Boc Group

0.5 g of the t-butoxycarbonyl-protected compound were dissolved in 50 ml CH₂Cl₂ ((abs.)) and mixed with 6 ml TFA at 0° C. The mixture was stirred with the disappearance of the educt molecules being monitored by thin-layer chromatography. Stirring continued for 1 hour after the educt had disappeared. The solvent was evaporated and the remaining residue shaken out with saturated NaHCO₃ and boiling acetic acid ethyl ester (10 times, 75 ml each). The organic phases were dried over magnesium sulphate followed by rotary evaporation.

### Example 2a

### N,N′-Bis-(1-t-butyloxycarbonyl-5-methyl-pyrazole-3-yl)-urea

1.22 g of 1 (6.2 mmol) were dissolved in 40 ml CH₂Cl₂ ((abs.)) and coupled according to general instruction A, however phosgene was applied as a 1.93M solution in toluene. The reaction product was re-crystallised from CH₂Cl₂:n-hexane (R_{F}=0.66 with CHCl₃:Methanol 30:1).

Yield: 1.1 g (2.6 mmol, 42%), melting point: decomposition at 161° C., ¹H-NMR (300 MHz, CDCl₃, 25° C.): δ=1.69 (s, 18H; 1), δ=2.28 (s, 6H; 2), δ=6.56 (s, 2H; 3), δ=9.73 (br, 2H; 4); ¹³C-NMR (50.4 MHz, CDCl₃, 25° C.): δ=14.4 (a), δ=28.0 (b), δ=86.5 (c), δ=96.8 (d), δ=141.8+147.3+151.0+153.6 (e, f, g, h); mass spectrum: (C₁, NH₃, 200° C.): m/z=421 (M+H⁺; 40%), 318 (M⁺-Boc (62%), 301 (100%); elementary analysis calculated for C₁₉H₂₈O₅N₆: C, 54.27; H, 6.71; N, 19.99; found C, 53.67; H, 6.87; N, 19.66.

### Example 2

### N,N′-Bis-(5-methyl-1H-pyrazole-3-yl)-urea

0.5 g of 2a (1.2 mmol) were dissolved in 50 ml CH₂Cl₂ ((abs.)) and converted according to general instruction B.

Yield: 0.12 g (0.55 mmol, 45%); melting point 202-206° C., ¹H-NMR (300 MHz, CDCl₃, 25° C.): δ=1.69 (s, 18H; 1), δ=2.28 (s, 6H; 2), δ=6.56 (s, 2H; 3), δ=9.73 (br, 2H; 4); ¹³C-NMR (50.4 MHz, methanol-d₄, 25° C.): δ=10.9 (a), δ=143.8+147.2+153.4 (b, c, d); mass spectrum (FAB+NBA): m/z=221 (M+H⁺; 62%) 137 (M⁺-pyrazole (70%), 51 (100%); high resolution mass spectroscopy (ESI-negative): m/z=220.1077 (measured), 220.1073 (calculated), 0.0004 (difference)

### Example 3a

### N,N′-Bis-(1-t-butyloxycarbonyle-5-methyl-pyrazole-3-yl)-oxalic acid diamide

2 g of 1 (10 mmol) were dissolved in 40 ml CH₂Cl₂ ((abs.)) and converted according to general instruction A. The product was re-crystallised from CH₂Cl₂ and the residue washed with cold n-hexane. (RF=0.75 with CHCl₃:methanol 30:1).

Yield: 1.1 g (5.6 mmol, 56%); melting point: decomposition 170° C., ¹H-NMR (300 MHz, CDCl₃, 25° C.): δ=1.71 (s, 18H; 1), δ=2.32 (s, 6H; 2), δ=6.81 (s, 2H; 3), δ=11.75 (br, 2H; 4); ¹³C-NMR: Due to the poor solubility and the strong quadruple moment of the nitrogen nuclei, it was impossible to receive a ¹³C-NMR spectrum. Mass spectrum (FD): m/z=448 (M⁺); elementary analysis calculated for C₂₀H₂₈O₆N₆: C, 53.56; H, 6.29; N, 18.74; found: C, 53.30; H, 6.22; N, 18.79.

### Example 3

### N,N′-Bis-(−5-methyl-1H-pyrazole-3-yl)-oxalic acid diamide

1 g of 3a (5.1 mmol) was dissolved in 50 ml CH₂Cl₂ ((abs.)) and converted according to general instruction B.

Yield: 0.3 g (1.2 mmol, 24%); melting point: >230° C.; ¹H NMR (300 MHz, DMSO-d₆, 25° C.): δ=2.37 (s, 6H; 1), δ=6.46 (s, 2H; 2), δ=10.70 (br, 2H; 3), δ=12.39 (br, 2H; 4); ¹³C NMR: Due to the poor solubility and the strong quadruple moment of the nitrogen nuclei, it was impossible to receive a ¹³C-NMR spectrum; mass spectrum (EI): m/z=248 (M⁺; 8%), 124 (31%), 97 (aminomethyl pyrazole; 100%); high resolution mass spectroscopy (EI): m/z=248.1017 (measured) 248.1022 (calculated) 0.0005 (difference).

### Example 4a

### N-N′-Bis-(1-t-butyloxycarbonyl-5-methyl-pyrazole-3-yl)-2,2-dimethyl-malonic acid diamide

1.5 g of 1 (in above figure)(7.6 mmol) were dissolved in 30 ml CHCl₃ ((abs.)) and converted according to general instruction A, but in this case, chloroform was heated to reflux before the acid chloride was added. The residue was re-crystallised from CH₂Cl₂:n-hexane (R_{F}=0.44 with CHCl₃:methanol (30:1).

Yield: 1 g (2 mmol, 27%); melting point: decomposition 124° C.; ¹H NMR (300 MHz, DMSO-d₆, 25° C.): δ=1.30 (s, 6H; 1), δ=1.43 (s, 18H; 2), δ=2.03 (s, 6H; 3), δ=6.43 (s, 2H; 4), δ=10.46 (s, 2H; 5); ¹³C NMR (50.4 MHz, CDCl₃, 25° C.): δ=14.4 (a), δ=27.8 (b), δ=27.9 (c), δ=51.4 (d), δ=86.5 (e), δ=98.5 (f), δ=150.7+151.4+153.2 (g, h, i), δ=169.2 (j); mass spectrum (FD): m/z=490 (M⁺; 14%), 390 (M⁺-Boc (75%), 290 (M⁺-2 Boc (100%).

### Example 4

### N-N′-Bis-(5-methyl-1H-pyrazole-3-yl)-2,2-dimethyl-malonic acid diamide

0.53 g of 4a (1.1 mmol) were dissolved in 30 ml CH₂Cl₂ ((abs.)) and converted according to general instruction B.

Yield: 97 mg (0.34 mmol, 31%); melting point: 204° C.; ¹H NMR (300 MHz, DMSO-d₆, 25° C.): δ=1.55 (s, 6H; 1), δ=2.25 (s, 6H; 2), δ=6.31 (s, 2H; 3), δ=9.85 (s, 2H; 4), δ=12.09 (s, 2H; 5); ¹³C NMR (50.4 MHz, methanol-d₄, 25° C.): δ=12.2 (a), δ=24.6 (b), δ=52.1 (c), δ=97.2 (d), δ=140.0+148.2+156.7 (e, f), δ=172.1 (g); mass spectrum (FD): m/z=290 (M⁺); high resolution mass spectroscopy (ESI-negative calculated M+Na⁺): m/z=313.1351 (measured), 313.1389 (calculated), 0.0038 (difference).

### Example 5a

### N-N′-Bis-(1-t-butyloxycarbonyl-5-methyl-pyrazole-3-yl)-terephthalic acid diamide

1 g of 1 (5.1 mmol) was dissolved in 30 ml CH₂Cl₂ ((abs.)) and converted according to general instruction A. The residue was re-crystallised from CHCl₃:n-hexane (R_{F}=0.79 with CH₂Cl₂:methanol (20:1).

Yield: 0.78 g (1.5 mol, 29%); Melting point: decomposition 181° C.; ¹H NMR (300 MHz, DMSO-d₆, 25° C.): δ=1.49 (s, 18H; 1), δ=2.19 (s, 6H; 2), δ=6.57 (s, 2H; 3), δ=8.02 (s, 4H; 4), δ=10.76 (s, 2H; 5); ¹³C NMR: Due to the poor solubility and the strong quadruple moment of the nitrogen nuclei, it was impossible to receive a ¹³C-NMR spectrum; mass spectrum (FD): m/z=524 (M⁺; 36%), 424 (M⁺-Boc (21%), 325 (M⁺-2 Boc (100%); elementary analysis calculated for C₂₆H₃₂O₆N₆: C, 59.53; H, 6.15; N, 16.02; measured: C, 59.20; H, 6.55; N, 15.89.

### Example 5

### N-N′-Bis-(5-methyl-1H-pyrazole-3-yl)-terephthalic acid diamide

0.5 g of 5a (0.95 mmol) were dissolved in 30 ml CH₂Cl₂ ((abs.)) and converted according to general instruction B.

Yield: 0.1 g (0.3 mmol, 33%); Melting point: >230° C.; ¹H NMR (500 MHz, DMSO-D₆, 25° C.): δ=2.35 (s, 6H; 1), δ=6.53 (s, 2H; 2), δ=8.18 (s, 4H; 3), δ=10.94 (s, 2H; 5), δ=12.24 (s, 2H; 5); ¹³C NMR: Due to the poor solubility and the strong quadruple moment of the nitrogen nuclei, it was impossible to receive a ¹³C-NMR spectrum; mass spectrum (EI): m/z=324 (M⁺; 13%), 228 (M⁺-aminopyrazole (34%), 206 (30%), 97 (aminopyrazole⁺ (35%), 44 (CO₂⁺); high resolution mass spectroscopy (EI): m/z=324.1333 (measured), 324.1335 (calculated), 0.0002 (difference).

### Example 6a

### N-N′-Bis-(1-t-butyloxycarbonyl-5-methyl-pyrazole-3-yl)-)-isophthalic acid diamide

2 g of 1 (10.2 mmol) were dissolved in 50 ml CH₂Cl₂ ((abs.)) and converted according to general instruction A, with the crude product shaken out with CHCl₃. The residue was re-crystallised from CHCl₃:n-hexane (R_{F}=0.21 with CH₂Cl₂:methanol (20:1).

Yield: 1.2 g (2.3 mmol, 22%); melting point: 171° C.; ¹H NMR (300 MHz, DMSO-d₆, 25° C.): δ=1.33 (s, 18H; 1), δ=1.98 (s, 6H; 2), δ=6.40 (s, 2H; 3), δ=7.60 (t, ³J(H, H)=7.6 Hz, 1H; 4), δ=7.93 (d, ³J(H, H)=7.3 Hz, 1H; 5), δ=8.23 (s, 1H; 6), δ=10.62 (s, 2H; 7); ¹³C NMR: Due to the poor solubility and the strong quadruple moment of the nitrogen nuclei, it was impossible to receive a ¹³C-NMR spectrum; mass spectrum (FD): m/z=524 (M⁺; 14%), 424 (M⁺-Boc (75%), 324 (M⁺-2 Boc (100%).

### Example 6

### N-N′-Bis-(5-methyl-1H-pyrazole-3-yl)-isophthalic acid diamide

0.5 g of 6a (0.95 mmol) were dissolved in 30 ml CH₂Cl₂ ((abs.)) and converted according to general instruction B.

Yield: 0.23 g (0.7 mmol, 75%); melting point: 172° C.; ¹H NMR (300 MHz, DMSO-d₆, 25° C.): δ=2.30 (s, 6H; 1), δ=6.50 (s, 2H; 2), δ=7.67 (t, ³J(H, H)=7.6 Hz, 1H; 3), δ=8.16 (d ³J(H, H)=8.0 Hz, 1H; 4), δ=8.61 (s, 2H; 5), δ=10.74 (s, 2H; 6), δ=12.21 (s, 2H; 7); ¹³C NMR (50.4 MHz, methanol-d₄, 25° C.): δ=12.2 (a), δ=97.5 (b), δ=128.1+130.0+132.1+135.6 (c, d, e, f), δ=165.1 (g); mass spectrum (EI): m/z=324 (M⁺; 53%), 228 (M⁺-aminopyrazole (100%), 206 (30%), 97 (aminopyrazole⁺ (23%); high resolution mass spectroscopy (ESI-negative): m/z=324.1328 (measured), 324.1335 (calculated), 0.0007 (difference).

### Example 7a

### N-N′-Bis-(1-t-butyloxycarbonyl-5-methyl-pyrazole-3-yl)-pyridine-2,6-dicarboxylic acid diamide

1 g of 1 (5.1 mmol) was dissolved in 50 ml CH₂Cl₂ ((abs.)) and converted according to general instruction A. The residue was re-crystallised from CH₂Cl₂:n-hexane (R_{F}=0.36 with acetate ester:n-hexane (2:1).

Yield: 0.5 g (0.95 mmol, 19%); melting point: decomposition 165° C.; ¹H NMR (500 MHz, CDCl₃, 25° C.): δ=1.57 (s, 18H; 1), δ=2.35 (s, 6H; 2), δ=6.82 (s, 2H; 3), δ=8.16 (t, ³J(H, H)=7.6 Hz, 1H; 4), δ=8.42 (d, ³J(H, H)=7.6 Hz, 1 H; 5), δ=12.02 (s, 2H; 6); ¹³C NMR: Due to the poor solubility and the strong quadruple moment of the nitrogen nuclei, it was impossible to receive a ¹³C-NMR spectrum; mass spectrum (FD): m/z=525 (M⁺; 100%), 425 (M⁺-Boc (12%), 325 (M⁺-2 Boc (38%).

### Example 7

### N-N′-Bis-(5-methyl-1H-pyrazol-3-yl)-pyridin-2,6-dicarboxylic acid amide

0.4 g of 7a (0.76 mmol) were dissolved in 30 ml CH₂Cl₂ ((abs.)) and converted according to general instruction B.

Yield: 0.12 g (0.37 mmol, 49%); melting point: 196° C.; ¹H NMR (500 MHz, DMSO-d₆, 25° C.): δ=2.20 (s, 6H; 1), δ=6.43 (s, 2H; 2), δ=8.16 (t, ³J(H, H)=7.0 Hz, 1H; 3), δ=8.24 (d ³J(H, H)=6.9 Hz, 1H; 4), δ=11.62 (s, 2H; 5); ¹³C NMR (50.4 MHz, DMSO-d₆, 25° C.): δ=12.2 (a), δ=97.9 (b), δ=126.4+140.0+150.4 (c, d, e), δ=162.8 (f); mass spectrum (CI, NH₃, 200° C.): m/z=326 (M+H⁺; 100%); high resolution mass spectroscopy (ESI-negative): m/z=325.1271 (measured) 325.1287 (calculated) 0.0016 (difference). Mass spectroscopy data see FIG. 1.

### Embodiment 2: Synthesis of 5-[(5-nitro-pyrazole-3-carbonyl)-amino]-pyrazole-3-carboxylic acid methyl ester, a Peptide-Like Bridged Heterocyclic Dimer of Type II

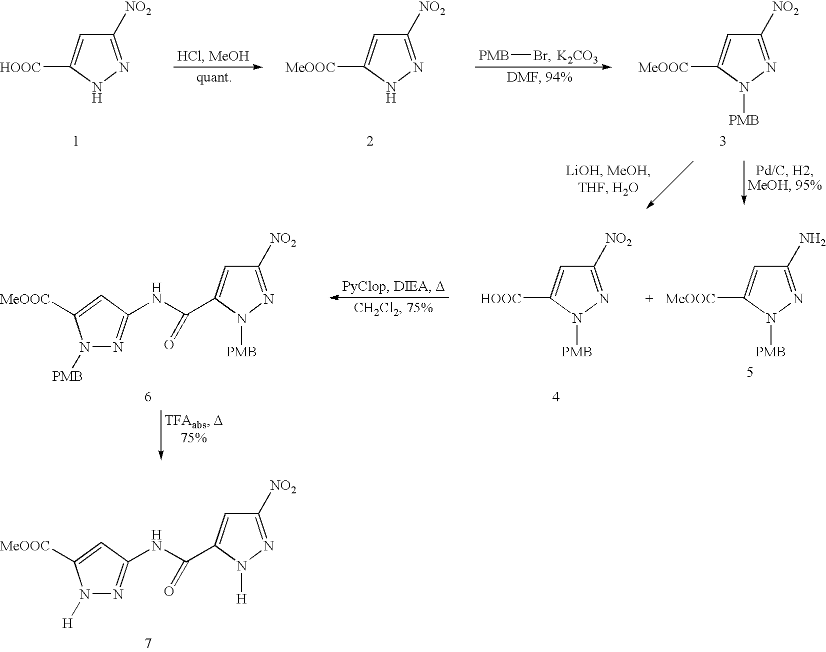

### Example 1

### 3-nitro-pyrazole-5-carboxylic acid methyl ester 2

100 ml of methanol, saturated with HCl, were added to a solution of 4.71 g (30.0 mmol, 1.00 eq) of the 3-nitro-pyrazole-5-carboxylic acid 1 in 200 ml methanol ((abs.)). The mixture was heated for reflux for 8 hours. Removal of the solvent yields 5.13 g (30 mmol, quant.) of a colourless solid.

(200 MHz, CDCl₃) δ=3.91 (s; 3H, CH₃), 7.52 (s; 1H, aromatic compounds (aromat.)CH) EA: C, 35.42; H, 3.33; N, 24.12.

### Example 2

### N-(4-methoxybenzyl)-3-nitro-pyrazole-5-carboxylic acid methyl ester 3

1.00 eq 3-nitro-pyrazole-5-carboxylic acid methyl ester of 2 in DMF ((abs.)) and 1.50 eq potassium carbonate were presented in a flask under argon. After addition of 1.20 eq 4-methoxy-benzylbromide the reaction mixture was stirred for 20 min at room temperature followed by stirring for 5 hours at 50° C. The mixture was then acidified with 1M HCl (up to pH=1). The aqueous phase was extracted with diethyl ether. Subsequently, the organic phase was washed with water and dried over magnesium sulphate. Removal of the solvent yields 94% of a yellowish solid.

¹H-NMR: Mixture of two regioisomers in a ratio of 5/1 (200 MHz, CDCl₃) δ=major: 3.78 (s; 3H, CH₃), 3.92 (s; 3H, CH₃), 5.76 (s; 2H, CH₂), 6.82-6.87, 7.33-7.39 (m; 5H, aromat. CH), minor: 3.81 (s; 3H, CH₃), 3.98 (s; 3H, CH₃); 5.80 (s; 2H, CH₂); 6.82-6.87, 7.33-7.39 (m; 5H, aromat. CH) EI: m/z=291 [M⁺]; 292 [M⁺+H]. EA: C, 53.64; H, 4.69; N, 14.30.

### Example 3

### N-(4-methoxybenzyl)-3-nitro-pyrazole-5-carboxylic acid 4

1.00 eq N-(4-methoxybenzyl)-3-nitro-pyrazole-5-carboxylic acid methyl ester 3 was dissolved in tetrahydrofuran (2 portions) and methanol (2 portions). 1 portion of water was added. 1.00 eq of lithium hydroxide was added at 0° C. The reaction mixture was stirred for 3 hours at room temperature, followed by removal of the solvent. The yellow solid was dissolved with water and acidified with 1M HCl. The precipitated colourless solid was filtered and dried.

¹H-NMR: Mixture of two regioisomers* (200 MHz, CDCl₃) δ=major: 3.78 (s; 3H, CH₃), 5.77 (s; 2H, CH₂), 6.82-6.87, 7.33-7.35 (m; 4H, aromat. CH), 7.51 (s; 1H, heterocyclic compounds (heterocy.) CH) EI: m/z=277 [M⁺].
¹ PMB=p-methoxybenzylbromide

### Example 4

### N-4-methoxybenzyl-3-amino-pyrazole-5-carboxylic acid methyl ester 5

1.00 eq N-(4-methoxybenzyl)-3-nitro-pyrazole-5-carboxylic acid methyl ester 3 was dissolved in a small amount of methanol; a spatula-tip of Pd/C (10%) was added. The reaction mixture was stirred under hydrogen atmosphere (balloon) for 5 hours at room temperature. The catalyst was filtered, the solvent removed and the product dried under an oil pump vacuum. The remaining residue yields 95% of a yellow solid.

¹H-NMR: Mixture of two regioisomers¹ (200 MHz, CDCl₃) δ=major: 3.72 (s; 3H, CH₃), 3.78 (s; 3H, CH₃), 5.54 (s; 2H, CH₂), 6.72-6.78, 7.16-7.20 (m; 4H, aromat. CH), 7.00 (s; 1H, heterocy. CH) EI: m/z=261 [M⁺], 292 [M⁺+H].
¹ In the following, only the main regioisomer is indicated.

### Example 5

### 2-(4-mMethoxy-benzyl)-5-{[(2-4-(Methoxy-benzyl)-5-nitro-pyrazole-3-carbonyl]-amino}-pyrazole-3-carboxylic acid methyl ester 6

1.00 eq N-(4-methoxybenzyl)-3-amino-pyrazole-5-carboxylic acid methyl ester 5, 1.00 eq N-(4-methoxybenzyl)-3-nitro-pyrazole-5-carboxylic acid 4, 1.30 eq PyClop and 3.90 eq diisopropylethylamine were dissolved in dichloromethane ((abs.)) and refluxed for approx. 9 hours. The solvent was then removed and the product purified chromatographically with silica gel and pentane/ethyl acetate. The purification yields 75% of a pale yellow solid.

¹H-NMR: Mixture of several regioisomers¹ (200 MHz, CDCl₃) δ=major: 3.78-3.79 (m; 6H, CH₃), 3.91 (s; 3H, CH₃), 5.59, 5.80 (s; 2H, CH₂), 6.80-6.84, 7.20-7.61 (m; 9H, aromat. CH), 8.34 (s; 1H. NH). FD: m/z=520 [M⁺]. HRMS (EI): 520,1708.
¹ In the following, only the main regioisomer is indicated.

### Example 6

### 5-[(5-nitro-pyrazole-3-carbonyl)-amino]-pyrazole-3 carboxylic acid methyl ester 7

1.00 eq 2-4-(methoxy-benzyl)-5-{[(2-4-(methoxy-benzyl)-5-nitro-pyrazole-3-carbonyl]-amino}-pyrazole-3-carboxylic acid methyl ester 6 was dissolved in trifluoroacetic acid ((abs.)) and refluxed overnight. After removal of the solvent, the remainder was washed with chloroform, water and pentane and subsequently dried. The reaction yielded 75% of a colourless solid.

¹H-NMR: (300 MHz, DMSOd₆) δ=3.86 (s; 3H, CH₃), 7.11 (s; 1H, heterocy. CH), 7.93 (s; 1H, heterocy. CH), 11.54 (s; 1H, NH), 13.9, 15.02 (s; 2H. heterocy. NH). EI: m/z=280 [M⁺], 281 [M+H⁺]. HRMS (ESI) 281.0628 (M+H⁺). Fp. 229° C.

### Embodiment 3: Synthesis of 5-{[5-({5-[(nitro-pyrazole-3-carbonyl)amino]-pyrazole-3-carbonyl}-amino]-pyrazole-3-carbonyl]-amino}pyrazole-3 carboxylic acid methyl ester, a peptide-like bridged heterocyclic dimer of type II

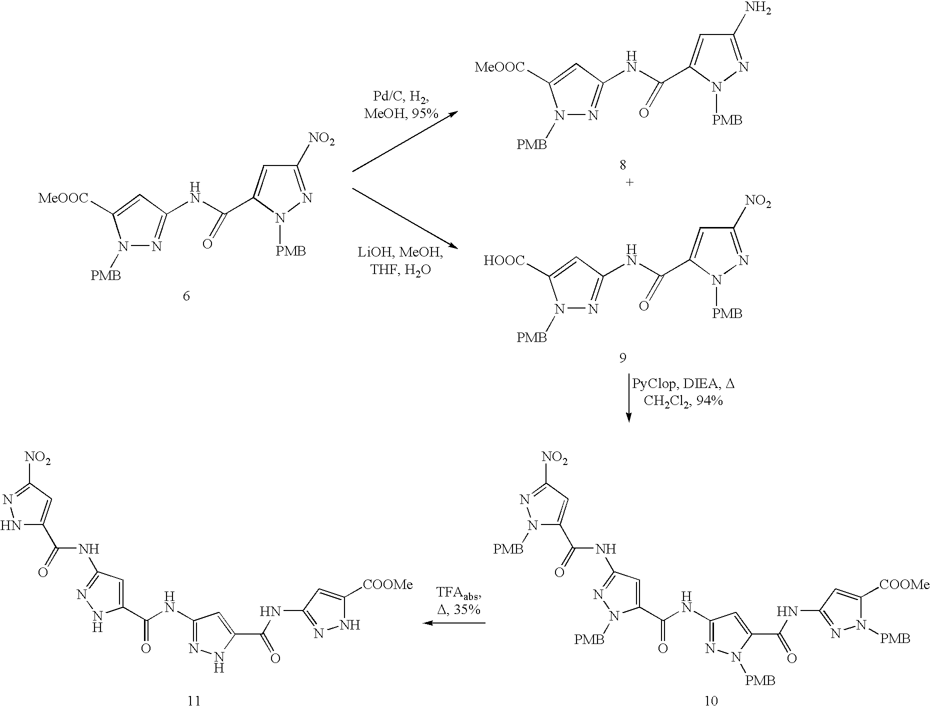

### Example 7

### 2-4-(methoxy-benzyl)-5-{[(2-4-(methoxy-benzyl)-5-amino-pyrazole-3-carbonyl]-amino}-pyrazole-3 carboxylic acid methyl ester 8

1.00 eq 2-4-(methoxy-benzyl)-5-{[(2-4-(methoxy-benzyl)-5-nitro-pyrazole-3-carbonyl]-amino}-pyrazole-3-carboxylic acid methyl ester 6 were dissolved in a small amount of dichloromethane and diluted with methanol before a spatula-tip of Pd/C (10%) was added. Afterwards the reaction mixture was stirred under a hydrogen atmosphere (balloon) for 5 hours at room temperature. After filtration of the catalyst and removal of the solvent, the product was dried under oil pump vacuum. The residue yields 98% of a yellow solid.

¹H-NMR: Mixture of several regioisomers¹ (200 MHz, CDCl₃) δ=major: 3.68-3.72 (m; 6H, CH₃), 3.82 (s; 3H, CH₃), 5.10, 5.55 (s; 4H, CH₂), 6.73-6.82, 7.10-7.38 (m; 10H, aromat. CH), 9.26 (s; 1H, NH). EI: m/z=490 [M⁺], 491 [M⁺+H].
¹ In the following only the main regioisomer was indicated.

### Example 8

### 2-4-(methoxy-benzyl)-5-{[(2-4-(methoxy-benzyl)-5-nitro-pyrazole-3-carbonyl]-amino}-pyrazole-3 carboxylic acid 9

1.00 eq 2-4-(methoxy-benzyl)-5-{[(2-4-(methoxy-benzyl)-5-nitro-pyrazole-3-carbonyl]-amino}-pyrazole-3 carboxylic acid methyl ester 6 were dissolved in tetrahydrofuran (2 portions) and methanol (2 portions), followed by the addition of 1 portion of water. 1 eq lithium hydroxide were added at 0° C. The reaction mixture was stirred for 3 hours at room temperature, and then the solvent was removed. The remaining yellow solid was dissolved with water and acidified with 1M HCl. The obtained colourless precipitate was filtered and dried, yielding 88% of a colourless solid.

¹H-NMR: Mixture of several regioisomers² (200 MHz, CDCl₃) δ=major: 3.71 (s; 3H, CH₃), 5.61-5,79 (s; 4H, CH₂), 6.76-7.52 (m; 10H, aromat. CH), 7.97 (s; 1H, NH). FD: m/z=506 [M⁺]. EA: C, 16.10; H, 57.08 N, 4.45.
² In the following only the main regioisomer was indicated.

### Example 9

### 1-(4-methoxy-benzyl)-3-({1-4-methoxy-benzyl)-5-[(1-(4-methoxybenzyl)-3-{[2-(4-methoxybenzyl)-5-nitro-pyrazole-3-carbonyl]-amino}-pyrazole-4-carbonyl)-amino]-pyrazol-3-carbonyl}-amino]-pyrazole-4-carboxylic acid methyl ester 10

1.00 eq 2-(4-methoxy-benzyl)-5-{[(2-4-(methoxy-benzyl)-5-amino-pyrazole-3-carbonyl]-amino}-pyrazole-3-carboxylic acid methyl ester 8, 1.00 eq 2-4-(methoxy-benzyl)-5-{[(2-4-(methoxy-benzyl)-5-nitro-pyrazole-3-carbonyl]-amino}-pyrazole-3-carboxylic acid 9, 1.30 eq PyClop and 3.90 eq diisopropylethylamine were dissolved in dichloromethane ((abs.)) and refluxed for about 9 hours. After removal of the solvent, the crude product was purified chromatographically with silica gel and pentane/ethyl acetate. The purification yields 94% of a light yellow solid.

¹H-NMR: Mixture of several regioisomers¹ (200 MHz, CDCl₃) δ=major: 3.58-3.68 (in each case; s 12H, CH₃), 3.78 (s; 3H, CH₃), 5.49-5.66 (4s; 8H, CH₂), 6.72-7.70 (m; 20H, aromat. CH), 8.41-8.57 (in each case; s 3H. NH). MALDI m/z=1002 [M+Na⁺]. Fp. 68° C.
¹In the following only the main regioisomer was indicated.

### Example 10

### 5-{[5-({5-[(nitro-pyrazole-3-carbonyl)amino]-pyrazole-3-carbonyl}-amino]-pyrazole-3-carbonyl[-amino}pyrazole-3-carboxylic acid methyl ester 11

1.00 eq 1-(4-methoxy-benzyl)-3-({1-4-methoxy-benzyl)-5-[(1-(4-methoxybenzyl)-3-{[2-(4-methoxy-benzyl)-5-nitro-pyrazole-3-carbonyl]-amino}-pyrazole-4-carbonyl)-amino]-pyrazole-3-carbonyl}-amino]-pyrazole-4-carboxylic acid methyl ester 10 was dissolved in trifluoroacetic acid and refluxed overnight in an argon atmosphere. After removal of the solvent, the residue was washed with dichloromethane, 25% ammonia and pentane and subsequently dried. The procedure yielded 35% of a slightly brownish solid.

¹H-NMR: (400 MHz, DMSOd₆)=(T=380 K) 3.89 (s; 3H, CH₃), 6.95, 7.12, 7.17, 7.55. (at each case; 4H, heterocy. CH), (T=300 K) 11.27 (br. s; 3H, NH), 11.54 (s; 1H, NH), 13.9, 15.02 (s; 2H. heterocy. NH). ESI_{neg.}: m/z=497 [M−H⁺].

### Practical Embodiment 4: Synthesis of 5-{[5-({5-[(nitro-pyrazole-3-carbonyl)amino]-pyrazole-3-carbonyl}-amino]-pyrazole-3-carboxylic acid methyl ester, a peptide-like bridged heterocyclic dimer of type II

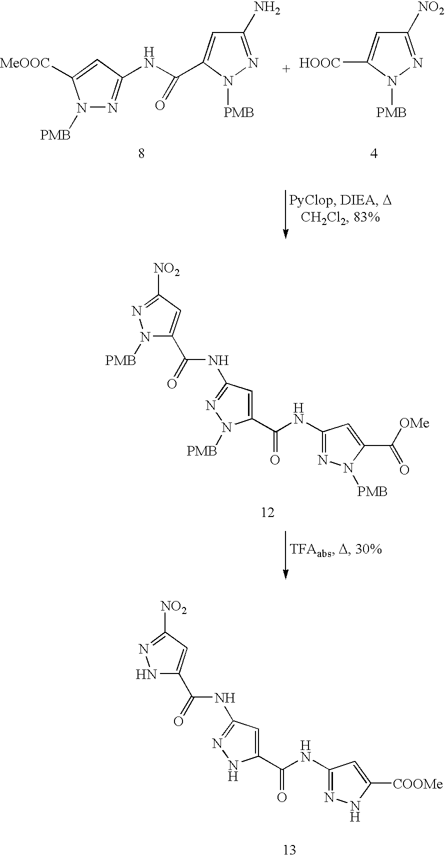

### Example 11

### 1-(4-methoxy-benzyl)-5-({2-(4-methoxy-benzyl)-5-{(1-(4-methoxy-benzyl)-5-nitro-pyrazole-3-carbonyl]-pyrazole-3-carbonyl]-amino}-pyrazole-3carbonyl-amino]pyrazole-3-carboxylic acid methyl ester 12

The synthesis was carried out in likeness to compound 10 and yielded 83% of a slightly yellowish solid.

¹H-NMR: Mixture of several regioisomers* (200 MHz, CDCl₃) δ=major: 3.66-3.69 (m; 9H, CH₃), 3.81 (s; 3H, CH₃), 5.53-5.69 (m; 6H, CH₂), 6.72-7.28 (m; 15H, aromat. CH), 8.41—8.57 (2s; 2H, NH). ESI m/z=749 [M⁺].

### Example 12

### 5-{[5-({5-[(nitro-pyrazole-3-carbonyl)amino]-pyrazole-3-carbonyl}-amino]-pyrazole-3-carboxylic acid methyl ester 13

The synthesis was carried out in likeness to compound 11 and yielded 30% of a colourless solid.

¹H-NMR: (300 MHz, DMSOd₆)=4.02 (s; 3H, CH₃), 7.2%, 7.74, 8.11, (3s; 3H, heterocy. CH), 11.41 (brs; 1H, NH), 11.61 (s; 1H, NH), 11.61 (brs; 1H, NH), 13.73 (brs; 2H. heterocy. NH), 15.15 (brs; 1H. heterocy. NH). FD: m/z=389 [M⁺]. HRMS (ESI) 390.956 [M+H⁺]. Fp. >230° C.

### Practical Embodiment 5: Synthesis of 2-(4-methoxy-benzyl)5-(2-{[2-(4-methoxy-benzyl)-5-nitro-pyrazole-3-carbonyl]-amino}-3-methyl-butyrylamino)-pyrazole-3-carboxylic acid methyl ester, a peptide-like bridged heterocyclic dimer of type II

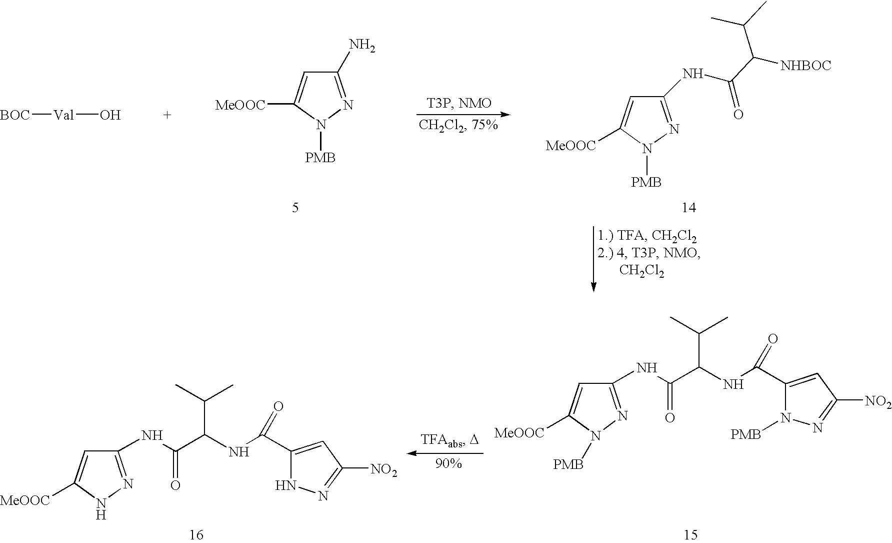

### Example 13

### 5-(2-tert.-butoxycarbonylamino-3-methyl-butyrylamino)-2-(4-methoxybenzyl)-pyrazole-3-carboxylic acid 14

1.00 eq N-4-methoxybenzyl-3-amino-pyrazole-5-carboxylic acid methyl ester 5, 1.10 eq BOC-Val-OH, 2.00 eq T3P and 6.00 eq N-methylmorpholine were stirred in dichloromethane ((abs.)) under argon atmosphere for 3 days, initially, on ice. After removal of the solvent, the crude product was purified chromatographically with silica gel and pentane/ethyl acetate. Purification yields 75% of a colourless solid.

¹H-NMR: (300 MHz, CDCl₃)=0.16 (s; 9H, CH₃), 0.83-0.94 (2d; 6H, CH₃), 1.18-1.22 (m; 1H, CH), 2.15-2.4 (m; 1H, CH), 3.70 (s; 3H, CH₃), 3.78 (s; 3H, CH₃), 5.52 (s; 2H, CH₂), 6.74-6.78 (m; 2H, aromatic CH), 7.12-7.16 (m; 2H, aromatic CH) 7.21 (s; 1H, heterocy. CH), 8.24 (s; 1H, NH). FD: m/z=460 [M⁺].

### Example 14

### 2-(4-methoxy-benzyl)5-(2-{[2-(4-methoxy-benzyl)-5-nitro-pyrazole-3-carbonyl]-amino}-3-methyl-butyrylamino)-pyrazole-3 carboxylic acid methyl ester 15

1.00 eq 5-(2-tert.-butoxycarbonylamino-3-methyl-butyrylamino)-2-(4-methoxybenzyl)-pyrazole-3 carboxylic acid 14 was stirred for 5 hours in an 1:1 mixture of dichloromethane and trifluoroacetic acid. The solvent was then removed. The remaining slightly pink solid was dried under an oil pump vacuum and dissolved in dichloromethane ((abs.)) under argon atmosphere. Subsequently, 1.00 eq N-4-methoxybenzyl-3-nitro-pyrazole-5 carboxylic acid, 2.00 eq T3P and 7.00 eq N-methylmorpholine were added and stirred for 3 days at room temperature, with stirring being performed initially, on ice. Chromatographical purification with silica gel and pentane/ethyl acetate yields 50% of a colourless solid.

¹H-NMR: (300 MHz, CDCl₃)=0.88-1.00 (m; 6H, CH₃), 1.18-1.22 (brs; 1H, CH), 3.63-3.69 (m; 6H, CH₃), 3.79 (s; 3H, CH₃), 4.40-4.45 (dd; 1H, CH), 5.45-5.73 (m; 5H, CH₂, NH), 6.65-7.24 (m; 10H, aromat. CH), 8.21 (s; 1H, NH). FD: m/z=619 [M⁺].

### Example 15

### 5-3-methyl-2{[(5-nitro-pyrazole-3-carbonyl)-amino]-butyrylamino}-pyrazole-3-carboxylic acid methyl ester 16

1.00 eq 2-(4-Methoxy-benzyl)5-(2-{[2-(4-methoxy-benzyl)-5-nitro-pyrazol-3-carbonyl]-amino}-3-methyl-butyrylamino)-pyrazl-3-carboxylic acid methylester 15 was dissolved in trifluoroacetic acid ((abs.)) and refluxed for 4 hours under argon atmosphere. Purification via column chromatography with silica gel and methanol/dichloromethane yields 90% of a colourless solid.

¹H-NMR: (300 MHz, DMSOd₆)=1.01-1.06 (t; 6H, CH₃), 2.12-2.14 (m; 1H, CH), 3.92 (s; 3H, CH₃), 4.16-4.57 (dd; 1H, CH), 7.09, 8.00 (2s; 2H, heterocy. CH), 8.94 (d; 1H, NH), 10.98 (2s; 1H, NH), 13.72, 14.86 (2s; 1H, heterocy. NH). FD: m/z=379 [M⁺]. HRMS (ESI) 380.1349 (M+H⁺). Fp. 217° C.

### Practical Embodiment 6: Synthesis of 5-{[5-hexanoylamino-2-(4-methoxy-benzyl-pyrazole-3-carbonyl]-amino}-2-(4-methoxy-benzyl)-pyrazole-3-carboxylic acid butylamide, a peptide-like bridged heterocyclic dimer of type II

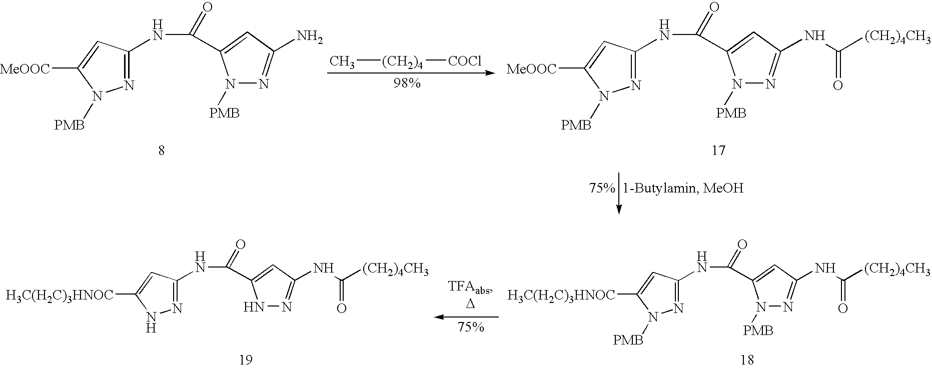

### Example 16

### 5-{[5-hexanoylamino-2-(4-methoxy-benzyl-pyrazole-3-carbonyl]-amino}-2-(4-methoxy-benzyl)-pyrazole-3-carboxlic acid methyl ester 17

1.00 eq 2-4-(methoxy-benzyl)-5-{[(2-4-(Methoxy-benzyl)-5-amino-pyrazole-3-carbonyl]-amino}-pyrazole-3-carboxylic acid methyl ester 8 was mixed with 1.00 eq hexanoic acid chloride and 1.50 eq diisopropylethylamine in tetrahydrofurane ((abs.)) and stirred overnight at room temperature under argon atmosphere. Subsequent chromatographical purification with silica gel and ethyl acetate/pentane yielded 98% of a colourless solid.

¹H-NMR: (300 MHz, CDCl₃) δ=major:1.03 (t; 3H, CH₃), 1.42-1.45 (m; 4H, CH₂), 1.77-1.82 (m; 2H, CH₂), 2.44 (t; 2H, CH₂), 3.91, 3.94 (2s; 6H, CH₃, 4.05 (s; 3H, CH₃), 5.77, 5.80 (2s; 4H, CH₂), 6.94-7.55 (m; 10H, aromat. CH), 8.52, 9.38 (2s; 2H, NH). EI: m/z=588 [M⁺] 589 [M+H⁺]. EA: C, 62.99; H, 6.29; N, 14.25.

### Example 17

### 5-{[5-hexanoylamino-2-(4-methoxy-benzyl-pyrazole-3-carbonyl]-amino}-2-(4-methoxy-benzyl)-pyrazole-3-carboxylic acid butylamide 18

1.00 eq 5-{[5-hexanoylamino-2-(4-methoxy-benzyl-pyrazole-3-carbonyl]-amino}-2-(4-methoxy-benzyl)-pyrazole-3-carboxylic acid methyl ester 17 was dissolved in an 1:1 mixture of methanol and butylamine and refluxed overnight in an argon atmosphere. Removal of the solvent and subsequent chromatographical purification with silica gel and ethyl acetate/pentane yielded 75% of a colourless solid.

¹H-NMR: (300 MHz, CDCl₃) δ=0.88-0.93 (m; 6H, CH₃), 1.21-2.25 (m; 10H, CH₂), 2.25 (t; 2H, CH₂), 3.30-3.37 (m; 2H, CH₂), 3.91, 3.94 (2s; 6H, CH₃), 5.54, 5.55 (2s; 4H, CH₂), 6.31 (t; 1H, NH), 6.71-7.27 (m; 10H, aromat. CH), 8.23, 9.10 (2s; 2H, NH).

### Example 18

### 5-[(5-hexanoylamino-pyrazole-3-carbonyl)amino]-pyrazole-3-carboxylic acid butylamide 19

1.00 eq 5-{[5-hexanoylamino-2-(4-methoxy-benzyl-pyrazole-3-carbonyl]-amino}-2-(4-methoxy-benzyl)-pyrazole-3-carboxylic acid butylamide 18 was dissolved in trifluoroacetic acid ((abs.)) and refluxed for 4 hours under an argon atmosphere. Removal of the solvent and subsequent chromatographical purification with silica gel and dichloromethane/methanol yielded 75% of a colourless solid.

¹H-NMR: (300 MHz, CDCl₃) δ=0.82-0.89 (m; 6H, CH₃), 1.25-1.57 (m; 10H, CH₂), 2.29 (t; 2H, CH₂), 3.19-3.31 (m; 2H, CH₂), 7. 19, 7.38 (2s; 2H, hetreocy. CH), 8.47 (brs; 1H, NH), 10.42, 10.93 (2s; 2H, NH), 13.10 (brs; 2H, heterocy. NH). HRMS (ESI) 390.2296 [M+H⁺].

Mass spectroscopy data (see FIG. 2).

### Results of Biophysical Measurements

To demonstrate the influence of the β-sheet binders described in the current invention, on the aggregation of amyloidogenic proteins, the following experimental approach, based on aminopyrazoles, was used.

Aβ(1-42) was used as the model molecule in the aggregation experiments. Aβ(1-42) is one component of the amyloid plaques, which are related with Alzheimer's disease (AD) (Glenner & Wong, 1984). In the early stages of Aβ, insoluble Aβ fibrils are produced. The results of several studies show that Aβ might have a causal role in the pathogenesis of AD (Selkoe, 1999). Apart from Aβ(1-42), the plaques consist of shorter amyloid fragments and there are several other synthetic Aβ fragments accessible and available. Of all the amyloid protein fragments described so far, Aβ(1-42) is the one with the fastest aggregation kinetics and it is the only one that includes the entire amino acid sequence. Hence, it can be assumed that if the aminopyrazoles disturb the Aβ(1-42) aggregation, they will disturb the aggregation of any other amyloid fragments as well.

The effect of aminopyrazoles on Aβ aggregation was tested during spontaneous multimerisation of the protein. The reaction was induced by reducing the solvent concentration from 100% DMSO (dimethylsulfoxide) to 5-15%. The samples were incubated for 12-72 hours.

In all experiments freshly diluted protein was compared with aggregated protein samples as well as with samples incubated in the presence of aminopyrazoles.

The inhibiting effect of the two most promising aminopyrazoles on Aβ aggregation was proved with two independent experimental methods in order to exclude systematic experimental errors.

By means of Fluorescence Correlation Spectroscopy (FCS) the diffusion time of a small molecule with fluorescence activity in a small volume can be measured. Using the correlation between diffusion time and molecular weight of the molecule, FCS measurements allow the determination of molecular weight. Hence, it is possible to measure the degree of multimerisation during aggregation studies. Two observations can be made during the aggregation process:
an increase in diffusion time and
an increase in fluorescence intensity

FIG. 3 illustrates the aminopyrazoles employed in the biophysical measurements.

FIG. 4 illustrates the differences between the FCS spectra (ConfoCor, Zeiss) of monomeric and aggregated Aβ(1-42). For each of the FIGS. 4 to 8, every sample was measured 10 times for 30 seconds; the resulting 10 plots were overlaid on one graph.

The influence of several aminopyrazoles on Aβ(1-42) aggregation is shown in FIGS. 5 to 8. These aminopyrazoles were dissolved in 100% DMSO. As the aminopyrazoles AmpOx and Trimer were not completely soluble in 100% DMSO and AmpiPht precipitated to 5% DMSO after dilution in 10 mM of sodium phosphate buffer pH 7.2; the exact concentration of these compounds in the aggregation assays is unknown. In order to eliminate particles, these dissolved solutions of aminopyrazoles were resolved in 10 mM of sodium phosphate buffer pH 7.2 to a final DMSO concentration of 5%. They were then filtered with Microcon YM 10 tubes. The aggregation assays contained 5.5-11 μM Aβ(1-42), mixed with 22 nM of oregon green-labelled Aβ(1-42) and, if indicated, 10 nM of aminopyrazole (referring to the initial solution) in 5-10% DMSO, 10 mM of sodium phosphate buffer pH 7.2. The reaction was incubated at room temperature, in the dark for 12-72 hours.

In all cases, addition of aminopyrazoles gave reproducible results of inhibition of Aβ(1-42) aggregation.

Precipitation and Differential Ultracentrifugation

Aβ(1-42) solutions were incubated under various conditions and their aggregation was analysed by a 100,000 g spin in an ultracentrifuge. Separate analysis of supernatant and pellet by means of tricine-SDS-polyacrylamide gel electrophoresis (Schägger & van Jagow, 1987) and subsequent silver staining of the protein bands (Heukeshoven & Derrick, 1985; Merril et al., 1981) revealed whether higher aggregates had been formed or not.

FIG. 9 illustrates that the amount of soluble Aβ (1-42) was reproducibly increased when the aggregation assays contained Trimer. After the 100,000 g centrifugation step, most of the protein was detected in the supernatant (S). If aggregation occurred without Trimer, most of the Aβ(1-42) formed insoluble aggregates. These aggregates precipitated during ultracentrifugation, thus most of the protein was located in the pellet fraction (P). Monomeric Aβ(1-42) is completely soluble and subsequently remains in the supernatant (S).

Adding AmpOx to the aggregation assays gave almost identical results (FIG. 10).

This differential ultracentrifugation technique has not previously been used to test other aminopyrazoles.

The results obtained with the FCS measurements are in good agreement with the results of the differential ultracentrifugation for both aminopyrazoles, Trimer and AmpOx. Thus, two independent experimental methods reproducibly demonstrated that the two β-sheet binders Trimer and AmpOx are able to disturb or inhibit the aggregation of Aβ(1-42).

The active agents based on the current invention may be used in the development of pharmaceuticals for patients in therapy, diagnostics and prophylaxis of diseases that are accompanied by the occurrence of abnormal protein structures. The expression “patient” refers to humans and vertebrates alike. Thus, application of the previously mentioned pharmaceuticals can be applied both in human and veterinary medicine. Pharmaceutically acceptable compositions of compounds according to the said claims are available in dimeric up to oligomeric form or as salts, esters, amides or prodrugs thereof; provided that reliable medical evaluations do not indicate exceeding toxicity, irritations or allergic reactions of the patient. The therapeutically active compounds resulting from this invention, may be applied to patients as part of a pharmaceutically acceptable composition, either in oral, rectal, parenteral, intravenous, intramuscular, subcutaneous, intracisternal, intravaginal, intraperitoneal, intravascular, intrathecal, intravesical, topical, local (powder, ointment or drops) or spray form (aerosol). Regular dosing or application intravenously, subcutaneously, intraperitoneally or intrathecally may be carried out by means of a pump or dosing unit. Pharmaceutical forms for local application of the compounds based on the current invention comprise of ointments, powders, suppositories, sprays, and a means for inhalation. To do this, the active compound is mixed under sterile conditions according to the respective requirements, with a physiologically active carrier, as well as possible preservatives, buffers, diluents and blowing agents.

### FIGURE INFORMATION AND LEGENDS

In the following, 10 figures are listed.

FIG. 1

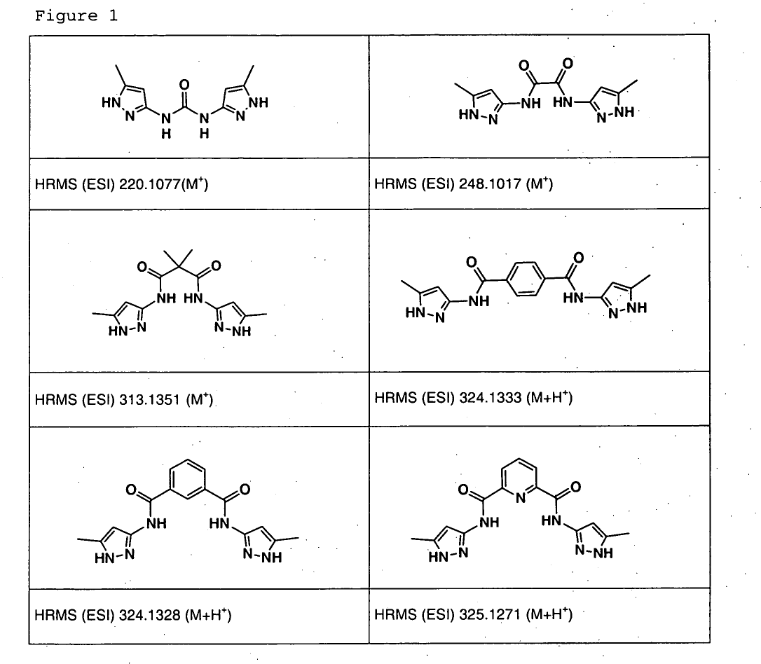

| | |
|---|---|
| HRMS: | High Resolution Mass Spectrometry |
| ESI: | Electro Spray Ionisation |
| M⁺: | Molecular ion |
| Top left: | Example 2 |
| Top right: | Example 3 |
| Middle left: | Example 4 |
| Middle right: | Example 5 |
| Bottom left: | Example 6 |
| Bottom right: | Example 7 |

FIG. 2

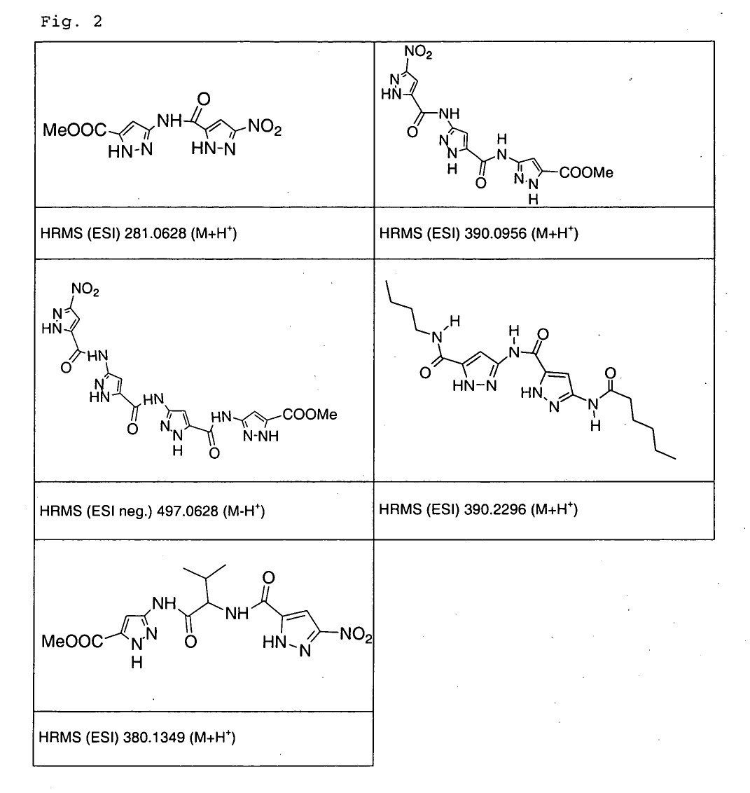

| | |
|---|---|
| HRMS: | High Resolution Mass Spectrometry |
| ESI: | Electro Spray Ionisation |
| M⁺: | Molecular ion |
| Top left: | Example 7 |
| Top right: | Example 13 |
| Middle left: | Example 11 |
| Middle right: | Example 19 |
| Bottom left: | Example 16 |

FIG. 3

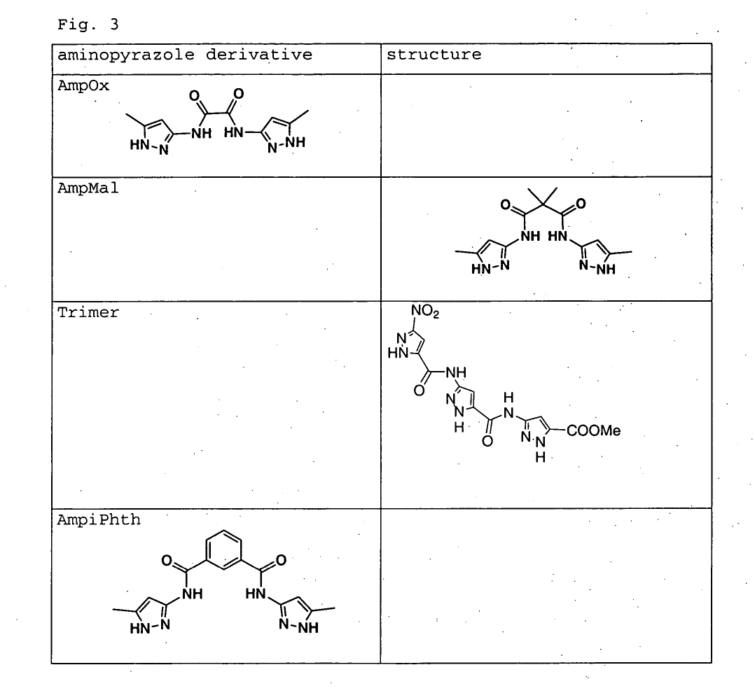

Aminopyrazole derivatives used for biophysical measurements: AmpOx=aminopyrazole dimer with oxalyl spacer (=compound no. 3, Type 1)

AmpMal=aminopyrazole dimer with malonyl spacer (=compound no. 4, Type 1)

Trimer=trimeric aminopyrazole carboxylic acid (one aminopyrazole molecule still bearing a nitro group), (=compound no. 13, Type 2)

AmpiPth=aminopyrazole dimer with one isophthaloyl spacer (=compound no. 6, Type 1)

FIGS. 4 to 10

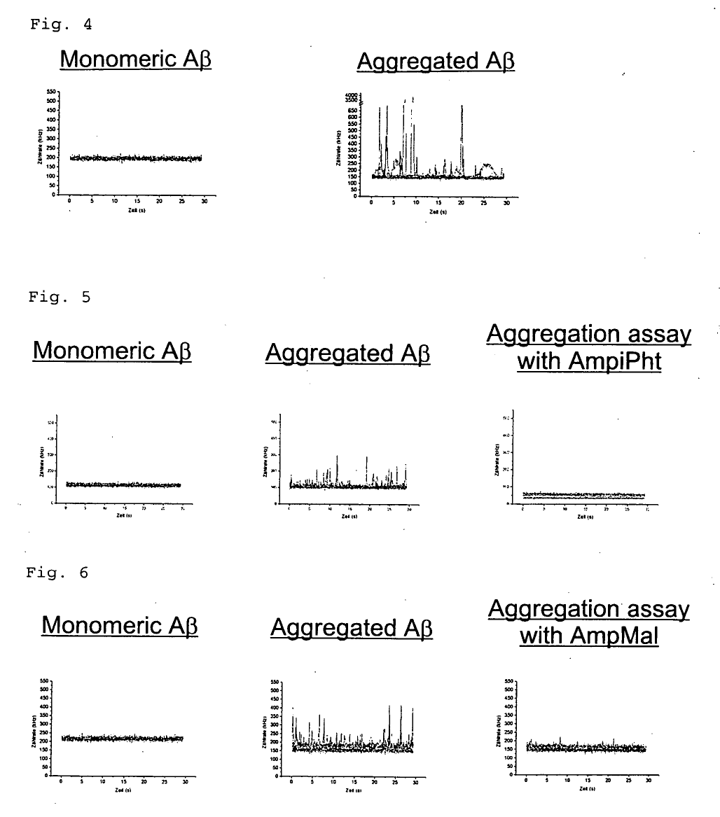

FIG. 4 illustrates the differences between the FCS spectra (ConfoCor, Zeiss) of monomeric and aggregated Aβ(1-42). Aβ is a component of the amyloid plaque, which is associated with the Alzheimer's disease (AD). For each of the FIGS. 4 to 8, every sample was measured 10 times during 30 seconds; the resulting 10 plots were overlaid in one graph. Aβ stands for β-amyloid plaque.

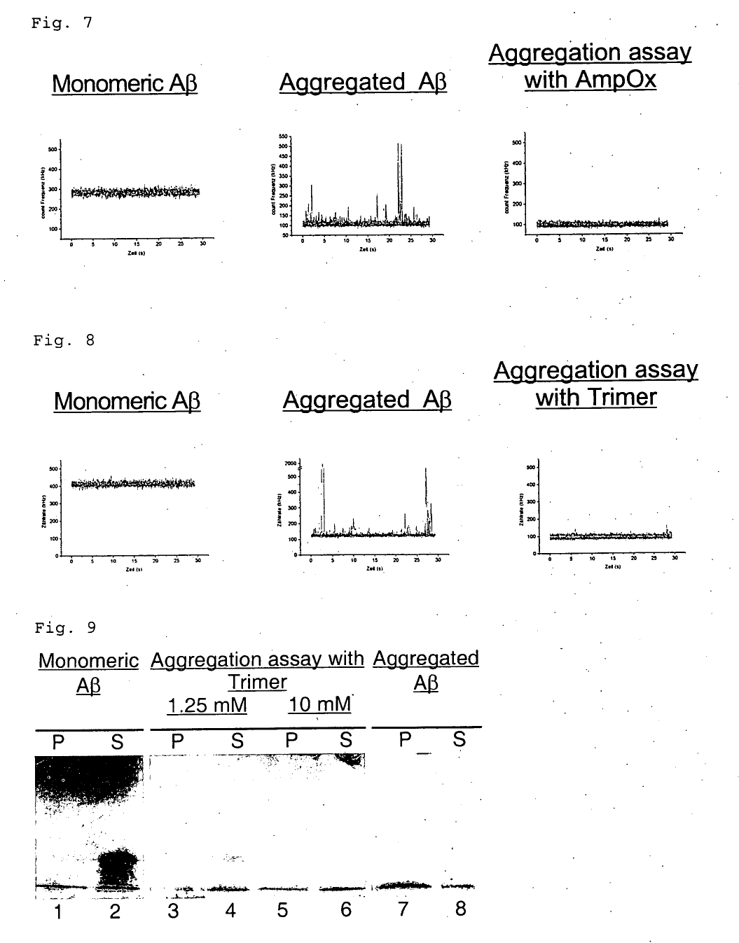

FIG. 4

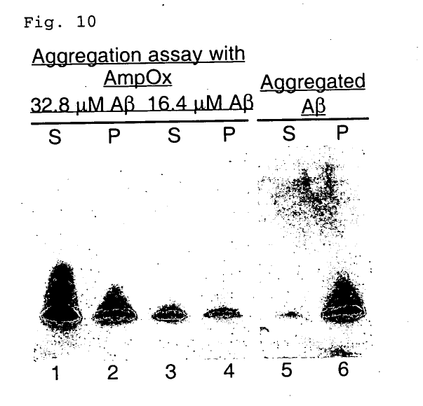

Left: 5.5 μM of Aβ(1-42) was mixed with 22 nM of oregon green labelled¹ Aβ(1-42). The spectrum was measured immediately after dilution of the Aβ(1-42) solution from 100% DMSO to 5% in 10 mM of sodium phosphate buffer pH 7.2.
¹ Oregon green labelled Aβ(1-42) was dissolved in 10 mM of sodium phosphate buffer pH 7.2, 200 mM NaCl and 0.2% SDS (sodium dodecyl sulphate) and diluted to 1:100 in the aggregation assay.

Right: 5.5 μM of Aβ(1-42) was mixed with 22 nM of oregon green labelled Aβ(1-42). The spectrum was measured 72 hours after dilution of the Aβ(1-42) solution from 100% DMSO to 5% in 10 mM of sodium phosphate buffer pH 7.2.

FIG. 5

Left: 5.5 μM of Aβ(1-42) was mixed with 22 nM of oregon green labelled Aβ(1-42). The spectrum was measured immediately after dilution of the Aβ(1-42) solution from 100% DMSO to 5% in 10 mM of sodium phosphate buffer pH 7.2.

Middle: 5.5 μM of Aβ(1-42) was mixed with 22 nM oregon green labelled Aβ(1-42). The spectrum was measured 18 hours after dilution of the Aβ(1-42) solution from 100% DMSO to 5% in 10 mM of sodium phosphate buffer pH 7.2.

Right: The assay was treated as described for the middle, additionally, 10 mM of AmpiPht was added to the aggregation assay.

FIG. 6

Left: [Sic: Diese Angabe fehlt im Deutschen Text.] 5.5 μM of Aβ(1-42) was mixed with 22 nM oregon green labelled Aβ(1-42). The spectrum was measured immediately after dilution of the Aβ(1-42) solution from 100% DMSO to 5% in 10 mM of sodium phosphate buffer pH 7.2.

Middle: [Sic: Diese Angabe fehlt im Deutschen Text.] 5.5 μM of Aβ(1-42) was mixed with 22 nM Oregon green labelled Aβ(1-42). The spectrum was measured 25 hours after dilution of the Aβ(1-42) solution from 100% DMSO to 5% in 10 mM of sodium phosphate buffer pH 7.2.

Right: The assay was treated as described for the middle; additionally, 10 mM of AmpMal was added to the aggregation assay.

FIG. 7

Left: 5.5 μM of Aβ(1-42) was mixed with 22 nM of oregon green labelled Aβ(1-42). The spectrum was measured immediately after dilution of the Aβ(1-42) solution from 100% DMSO to 5% in 10 mM of sodium phosphate buffer pH 7.2

Middle: 5.5 μM of Aβ(1-42) was mixed with 22 nM of oregon green labelled Aβ(1-42). The spectrum was measured 25 hours after dilution of the Aβ(1-42) solution from 100% DMSO to 5% in 10 mM of sodium phosphate buffer pH 7.2

Right: The assay was treated as described for the middle; additionally, 10 mM of AmpOx was added to the aggregation assay.

FIG. 8

Left: 5.5 μM of Aβ(1-42) was mixed with 22 nM of oregon green labelled Aβ(1-42). The spectrum was measured immediately after dilution of the Aβ(1-42) solution from 100% DMSO to 10% in 10 mM of sodium phosphate buffer pH 7.2.

Middle: 5.5 μM of Aβ(1-42) was mixed with 22 nM of oregon green labelled Aβ(1-42). The spectrum was measured 48 hours after dilution of the Aβ(1-42) solution from 100% DMSO to 10% in 10 mM of sodium phosphate buffer pH 7.2.

Right: The assay was treated as described for the middle; additionally, 10 mM of Trimer were added to the aggregation assay.

FIG. 9

Distributions of Aβ(1-42) between supernatant (S) and pellet (P) after a 100,000 g centrifugation. The Aβ(1-42) dissolved in 100% DMSO was diluted to a concentration of 11 μM in 10 mM of sodium phosphate buffer pH 7.2. All samples contained 22 nM of oregon green labelled Aβ(1-42) and were incubated for 48 hours at room temperature in 10% DMSO.

Lines 1 and 2: monomeric Aβ, centrifuged immediately after dilution

Lines 3 and 4: aggregated Aβ with 1.25 mM of Trimer in the aggregation assay

Lines 5 and 6: aggregated Aβ with 10 mM Trimer in the aggregation assay

Lines 7 and 8: aggregated Aβ without aminopyrazoles in the aggregation assay

FIG. 10

Distribution of Aβ(1-42) between supernatant (S) and pellet (P) after a 100,000 g centrifugation. Aβ(1-42) was dissolved in 100% DMSO and diluted to a concentration of 32.8 μM and 16.4 μM in 10 mM of sodium phosphate buffer pH 7.2. The protein was incubated at room temperature for 6 h in 6% DMSO with and without 20 mM AmpOx.

Lines 1 and 2: 32.8 μM of aggregated Aβ(1-42) with 20 mM of AmpOx in the aggregation assay

Lines 3 and 4: 16.4 μM of aggregated Aβ(1-42) with 20 mM of AmpOx in the aggregation assay

Lines 5 and 6: 16.4 μM of aggregated Aβ without aminopyrazoles in the aggregation assay.

1. Active agents for preventing the formation of β-amyloid plaques and for dissolving already pre-existing ones, wherein said active agents feature a linear DAD-structure (donor-acceptor-donor) with donor-acceptor distances of 3.5-4.0 Å and acceptor-donor distances of 2.6-2.9 Å, so that they recognise and bind β-sheet structures of peptides or proteins.

2. A compound of the formula 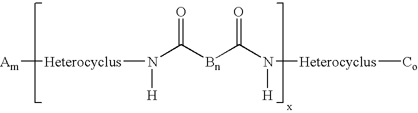
Type I
Wherein
A,C=Rest (linear, branched or cyclic alkyl, alkenyl or alkynyl with or without OH substituents or halides, phenyl, phenylalkyl, phenylalkenyl, phenylalkynyl, phenylcycloalkyl, phenylcycloalkenyl, phenylcycloalkynyl, cycloalkyl-alkyl, cycloalkyl-alkenyl, cycloalkyl-alkynyl, heterocyclo-alkyl, heterocyclo-alkenyl, heterocyclo-alkynyl, acyl, aryl, aryloxy, heteroaryl, heteroaryloxy, aroyl, benzyl, (aryl)alkyloxycarbonyl, linear or branched alkoxyalkyl with or without OH substituents, polyethoxy-alkyl, polyethoxy-alkenyl, polyethoxy-alkynyl, polyethoxy-cycloalkyl, polyethoxy-cycloalkenyl, polyethoxy-cycloalkynyl, polyethoxy-aryl, polyethoxy-alkyl-aryl, polyethoxy-heterocycloalkyl, polyethoxy-heterocycloaryl, primary, secondary, tertiary or quaternary ammonium, amino-alkyl, amino-alkenyl, amino-alkynyl, amino-cycloalkyl, amino-alkyl-cycloalkyl, amino-cycloalkyl-alkyl, amino-phenyl, amino-alkyl-phenyl, amino-phenyl-alkyl, all hydroxylamines, all cyano compounds, all nitriles and isonitriles, all halides thereof, formyl, alkanal, alkenal, alkynal, cycloalkenal, benzyl carbaldehyde, heteroaryl-carbaldehyde, benzyl-alkyl-carbaldehyde, heteroaryl-carbaldehyde, aliphatic heteroalkyl-alkenal (-alkenyl-alkenal, alkynyl-alkenal), alkanone, alkenone, alkynone, cycloalkyl-alkanone, dicycloalkanone, arylalkanone, heteroaryl-alkanone, nitro, alkylsulfoxy, alkylsulfonyl, CONH₂, CONHR, CONR₂, all imines, all oximes, all hydrazones, CH═NOR, thio, thio-alkyl, thio-alkenyl, thio-alkynyl, thio-cycloalkyl, thio-alkyl-cycloalkyl, thio-cycloalkyl-alkyl, thio-phenyl, thio-alkyl-phenyl, thio-phenyl-alkyl, alkylthio, halide, hydroxy, hydroxy-alkyl, hydroxy-alkenyl, hydroxy-alkynyl, hydroxy-cycloalkyl, hydroxy-alkyl-cycloalkyl, hydroxy-cycloalkyl-alkyl, hydroxy-phenyl, hydroxy-alkyl-phenyl, hydroxy-phenyl-alkyl, alkoxy, alkoxycarbonyl, alkyl-, alkenyl, alkynyl-, aryl-, heteroalkyl-, heteroaryl-carboxylic acids, and all esters as well as carboxylic acid amides thereof, linear or branched alkylsulphonate, alkenylsulphonate, alkynylsulphonate, linear or branched alkylbenzenesulphonate, alkenylbenzenesulphonate, alkynylbenzenesulphonate, aminosulphonyl-alkyl, aminosulphonyl-alkenyl, aminosulphonyl-alkynyl, aminosulphonyl-cycloalkyl, aminosulphonyl-cycloalkenyl, aminosulphonyl-cycloalkynyl, linear or branched alkyl-sulphonamide, alkenyl-sulphonamide, alkynyl-sulphonamide, cycloalkyl-sulphonamide, cycloalkenyl-sulphonamide, cycloalkynyl-sulphonamide, phenyl-sulphonamide, heterocyclo-sulphonic acid, heterocyclo-sulphonamide, heterocyclo-alkyl-sulphonic acid, heterocyclo-alkyl-sulphonic acid, heterocyclo-alkenyl-sulphonic acid, heterocyclo-alkenyl-sulphonamide, heterocyclo-alkynyl-sulphonic acid, heterocyclo-alkynyl-sulphonic acid, aryl-sulphonic acid, aryl-sulphonamide, aryl-alkyl-sulphonic acid, aryl-alkyl-sulphonamide, aryl-alkenyl-sulphonic acid, aryl-alkenyl-sulphonamide, aryl-alkynyl-sulphonic acid, aryl-alkynyl-sulphonamide, alkyl-, alkenyl, alkynyl-, aryl-, heteroalkyl-, heteroaryl-phosphonic acids as well as all esters and amides thereof, alkyl-, alkenyl, alkynyl-, aryl-, heteroalkyl-, heteroaryl-phosphinic acids as well as all esters and amides thereof), comply with donor-acceptor distances of 3.5-4.0 Å and acceptor-donor distances of 2.6-2.9 Å, so that they recognise and bind β-sheet structures of peptides or proteins.

3. A compound according to claim 2, wherein said spacer B is a linear or branched alkyl, alkenyl, alkynyl, halide, aryl, aryl(di)oxy, heteroaryl, heteroaryl(di)oxy, benzyl, xylyl, linear or branched alkoxyalkyl, oligo(ethyleneglycol), amino, alkylamino, alkoxy or hydrazino group and complies with said donor-acceptor and said acceptor-donor-distances.

4. A compound according to claim 2, wherein said heterocycle represents a substituted aromatic compound that complies with said donor-acceptor and said acceptor-donor-distances.

5. A compound according to claim 2, wherein said heterocyclic compound represents an aminopyrazole, aminotriazole, diaminoquinolone, maleic acid hydrazide, xanthine isomer or hydrazinomethoxybenzamide or a salt of a pharmaceutically acceptable acid or a prodrug thereof, complies with said donor-acceptor and said acceptor-donor-distances.

6. A compound of the formula 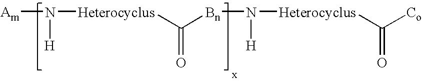
Wherein
A,C=Rest (linear, branched or cyclic alkyl, alkenyl or alkynyl with or without OH substituents or halides, phenyl, phenylalkyl, phenylalkenyl, phenylalkynyl, phenylcycloalkyl, phenylcycloalkenyl, phenylcycloalkynyl, cycloalkyl-alkyl, cycloalkyl-alkenyl, cycloalkyl-alkynyl, heterocyclo-alkyl, heterocyclo-alkenyl, heterocyclo-alkynyl, acyl, aryl, aryloxy, heteroaryl, heteroaryloxy, aroyl, benzyl, (aryl)alkyloxycarbonyl, linear or branched alkoxyalkyl with or without OH substituents, polyethoxy-alkyl, polyethoxy-alkenyl, polyethoxy-alkynyl, polyethoxy-cycloalkyl, polyethoxy-cycloalkenyl, polyethoxy-cycloalkynyl, polyethoxy-aryl, polyethoxy-alkyl-aryl, polyethoxy-heterocycloalkyl, polyethoxy-heterocycloaryl, primary, secondary, tertiary or quaternary ammonium, amino-alkyl, amino-alkenyl, amino-alkynyl, amino-cycloalkyl, amino-alkyl-cycloalkyl, amino-cycloalkyl-alkyl, amino-phenyl, amino-alkyl-phenyl, amino-phenyl-alkyl, all hydroxylamines thereof, all cyano compounds thereof, all nitriles and isonitriles, all halides, formyl, alkanal, alkenal, alkynal, cycloalkenal, benzyl carbaldehyde, heteroaryl-carbaldehyde, benzyl-alkyl-carbaldehyde, heteroaryl-carbaldehyde, aliphatic heteroalkyl-alkenal (-alkenyl-alkenal, alkynyl-alkenal), alkanone, alkenone, alkynone, cycloalkyl-alkanone, dicycloalkanone, arylalkanone, heteroaryl-alkanone, nitro, alkylsulfoxy, alkylsulfonyl, CONH₂, CONHR, CONR₂, all imines, all oximes, all hydrazones, CH═NOR, thio, thio-alkyl, thio-alkenyl, thio-alkynyl, thio-cycloalkyl, thio-alkyl-cycloalkyl, thio-cycloalkyl-alkyl, thio-phenyl, thio-alkyl-phenyl, thio-phenyl-alkyl, alkylthio, halide, hydroxy, hydroxy-alkyl, hydroxy-alkenyl, hydroxy-alkynyl, hydroxy-cycloalkyl, hydroxy-alkyl-cycloalkyl, hydroxy-cycloalkyl-alkyl, hydroxy-phenyl, hydroxy-alkyl-phenyl, hydroxy-phenyl-alkyl, alkoxy, alkoxycarbonyl, alkyl-, alkenyl, alkynyl-, aryl-, heteroalkyl-, heteroaryl-carboxylic acids, all esters as well as carboxylic amides thereof, linear or branched alkylsulphonate, alkenylsulphonate, alkynylsulphonate, linear or branched alkylbenzenesulphonate, alkenylbenzenesulphonate, alkynylbenzenesulphonate, aminosulphonyl-alkyl, aminosulphonyl-alkenyl, aminosulphonyl-alkynyl, aminosulphonyl-cycloalkyl, aminosulphonyl-cycloalkenyl, aminosulphonyl-cycloalkynyl, linear or branched alkyl-sulphonamide, alkenyl-sulphonamide, alkynyl-sulphonamide, cycloalkyl-sulphonamide, cycloalkenyl-sulphonamide, cycloalkynyl-sulphonamide, phenyl-sulphonamide, heterocyclo-sulphonic acid, heterocyclo-sulphonamide, heterocyclo-alkyl-sulphonic acid, heterocyclo-alkyl-sulphonic acid, heterocyclo-alkenyl-sulphonic acid, heterocyclo-alkenyl-sulphonamide, heterocyclo-alkynyl-sulphonic acid, heterocyclo-alkynyl-sulphonic acid, aryl-sulphonic acid, aryl-sulphonamide, aryl-alkyl-sulphonamide, aryl-alkyl-sulphonamide, aryl-alkenyl-sulphonic acid, aryl-alkenyl-sulphonamide, aryl-alkynyl-sulphonic acid, aryl-alkynyl-sulphonamide, alkyl-, alkenyl, alkynyl-, aryl-, heteroalkyl-, heteroaryl-phosphonic acids and all esters and amides thereof, alkyl-, alkenyl, alkynyl-, aryl-, heteroalkyl-, heteroaryl-phosphinic acids and all esters as well as amides thereof) comply with donor-acceptor distances of 3.5-4.0 Å and acceptor-donor distances of 2.6-2.9 Å so that they recognise and bind β-sheet structures of peptides or proteins.

7. A compound according to claim 6, wherein B represents an amino group (NH) and/or any natural or unnatural α- or β-amino acid in its (D)- or (L)-configuration and complies with said donor-acceptor and said acceptor-donor-distances.

8. A compound according to claim 6, wherein said heterocycle represents a substituted aromatic compound, complying with said donor-acceptor and said acceptor-donor-distances.

9. A compound according to claim 6, wherein said heterocycle represents an aminopyrazole, aminotriazole, azacyclohexanone, maleic acid hydrazide, xanthine isomer, hydrazinomethoxybenzamide or tetrahydropyridazinone or a salt of a pharmaceutically acceptable acid and a prodrug thereof, complies with said donor-acceptor and said acceptor-donor-distances.

10. A method for the production of diacyl-bridged heterocyclic compounds of type I according to claim 2, characterised by the following steps:
masking of heterocyclic amines or diamines as educts by appropriate protecting groups
conversion of the educts with an acid chloride and a sterically demanding base
stirring overnight
removal of all protecting groups
purification of the product

11. A method for the production of diacyl-bridged heterocyclic compounds of type I according to claim 10, wherein a symmetrical final product is formed through
application of the educts and the acid chloride in a ratio of 2:1
stirring overnight at room temperature

12. A method for the production of diacyl-bridged heterocycles of type I according to claim 10, wherein an asymmetrical dimeric product is formed, whereby:
the educt and the acid chloride are stirred in a ratio of 1:1 overnight on ice
a subsequent second conversion is performed, by adding an excess of another educt and reacting through stirring under reflux overnight

13. A method for the production of diacyl-bridged heterocycles of type I according to claim 10, wherein oligomeric final products are formed, whereby:
masked diamines are applied as educts
the production steps are repeated step by step until the desired chain length is achieved

14. A method for the production of peptide-like heterocycles of type II according to claim 6, characterised by the following steps:
the masking of the amino acids
conversion of the amino acids with a peptide coupling reagent
deprotection
purification of the products

15. A method for the production of peptide-like heterocycles of type II according to claim 14, wherein oligomeric final products are formed.

16. Utilisation of the compounds according to claim 1 for the manufacturing of pharmaceuticals for diagnosis, therapy and prophylaxis of diseases associated with abnormal β-sheet structures and subsequent abnormal protein aggregation.

17. Utilisation of compounds according to claim 1, wherein said compounds can be applied for the treatment of Alzheimer's disease and prion-associated diseases.

18. Utilisation of compounds according to claim 1, wherein said compounds can be applied by the following methods: intravenously, subcutaneously, intraperitoneally, intrathecally, intravesically, topically or as an aerosol.

19. A compound according to claim 2, wherein said compound is produced according to claim 10.

20. A compound according to claim 6, wherein said compound is produced according to claim 14.

21. A method for the production of diacyl-bridged heterocycles of type I according to claim 11, wherein an asymmetrical dimeric product is formed, whereby:
the educt and the acid chloride are stirred in a ratio of 1:1 overnight on ice
a subsequent second conversion is performed, by adding an excess of another educt and reacting through stirring under reflux overnight

22. A method for the production of diacyl-bridged heterocycles of type I according to claim 11, wherein oligomeric final products are formed, whereby:
masked diamines are applied as educts
the production steps are repeated step by step until the desired chain length is achieved

23. A method for the production of diacyl-bridged heterocycles of type I according to claim 12, wherein oligomeric final products are formed, whereby:
masked diamines are applied as educts
the production steps are repeated step by step until the desired chain length is achieved

24. Utilisation of the compounds according to claim 2 for the manufacturing of pharmaceuticals for diagnosis, therapy and prophylaxis of diseases associated with abnormal β-sheet structures and subsequent abnormal protein aggregation.

25. Utilisation of compounds according to claim 2, wherein said compounds can be applied for the treatment of Alzheimer's disease and prion-associated diseases.

26. Utilisation of compounds according to claim 2, wherein said compounds can be applied by the following methods: intravenously, subcutaneously, intraperitoneally, intrathecally, intravesically, topically or as an aerosol.

27. Utilisation of the compounds according to claim 6 for the manufacturing of pharmaceuticals for diagnosis, therapy and prophylaxis of diseases associated with abnormal β-sheet structures and subsequent abnormal protein aggregation.

28. Utilisation of compounds according to claim 6, wherein said compounds can be applied for the treatment of Alzheimer's disease and prion-associated diseases.

29. Utilisation of compounds according to claim 6, wherein said compounds can be applied by the following methods: intravenously, subcutaneously, intraperitoneally, intrathecally, intravesically, topically or as an aerosol.

30. A compound according to claim 2, wherein said compound is produced according to claim 11.

31. A compound according to claim 2, wherein said compound is produced according to claim 12.

32. A compound according to claim 2, wherein said compound is produced according to claim 13.

33. A compound according to claim 6, wherein said compound is produced according to claim 15.
